(19) United States
(12) Patent Application Publication
(10) Pub. No.: US 2014/0364335 A1
(43) Pub. Date: Dec. 11, 2014

(54) METHOD FOR SCREENING FOR NRF1 REGULATOR

(71) Applicants: Mochida Pharmaceutical Co., Ltd., Shinjuku-ku (JP); Tohoku University, Sendai-shi (JP)

(72) Inventors: Masayuki Yamamoto, Miyagi (JP); Yuki Furusawa, Tokyo (JP)

(21) Appl. No.: 14/373,692

(22) PCT Filed: Jan. 23, 2013
(86) PCT No.: PCT/JP2013/051941

§ 371 Date: Jul. 22, 2014

(30) Foreign Application Priority Data
Jan. 24, 2012 (JP) 2012-011833

(51) Int. Cl. G01N 33/50

(52) U.S. Cl. CPC G01N 33/5032; G01N 2500/04
USPC 506/9; 435/6.12; 435/8

(57) ABSTRACT

The present invention provides a method for screening a test compound for a compound regulating Nrf1 activity, comprising using Nrf1 and Lipin1 and/or PGC-1β. A method for screening for a compound regulating Nrf1 activity is provided by the invention.

### TECHNICAL FIELD

The present invention relates to a method for screening for a compound regulating Nrf1 activity.

### BACKGROUND ART

The liver is the central organ for the metabolism of fatty acids, which are metabolized by triglyceride synthesis or oxidative metabolism. These metabolic processes are in part regulated at the transcriptional level, and dysregulation leads to pathological changes. The most common hepatic alteration is fatty degeneration, which is associated with a progressive cascade of lipid disorders, such as hepatic steatosis, non-alcoholic steatohepatitis (NASH), cirrhosis, and eventually hepatocellular carcinoma. Understanding the transcriptional network of lipid metabolism will provide new insights into the pathogenesis of these hepatic disorders.

In the past several decades, various transcription factors have been found to be implicated in hepatic lipid metabolism. For example, peroxisome proliferator-activated receptor alpha (PPARα) has an important role in fatty acid degradation. In response to ligands, such as free fatty acids, PPARα forms a complex with its coactivator and subsequently induces the expression of genes involved in lipid metabolic processes, such as fatty acid oxidation. In PPARα knockout mice, impaired mitochondrial n-oxidation and fasting-induced hepatic steatosis are observed. In addition, sterol regulatory element-binding proteins (SREBPs) are key regulators of lipid synthesis. Transgenic mice expressing a constitutively active form of SREBP1 develop hepatic steatosis. Moreover, emerging evidence suggests that hepatic lipid metabolism is also regulated by various stress-inducible transcription factors, such as hypoxia-inducible factors and endoplasmic reticulum stress-induced transcription factors. Therefore, it seems that energy metabolism is elaborately coupled with the adaptive stress response.

NF-E2-related factor 1 (Nrf1) and Nrf2 are members of the Cap'n'collar (CNC) transcription factor family, which have been characterized as regulators of antioxidative and xenobiotic detoxifying enzyme genes. Under static conditions, Nrf2 is rapidly ubiquitinated by the Keap1-Cul3 E3 ubiquitin ligase complex and degraded through ubiquitin-proteasome pathway. Upon exposure to electrophiles or oxidative stresses, Nrf2 accumulates in nucleus where it forms a heterodimer with small Maf proteins and activates gene expression through binding to the antioxidant/electrophile response element (ARE/EpRE). Nrf2 knockout mice show an increased susceptibility to stresses due to the impaired induction of ARE-dependent cytoprotective genes (Literature 1). Recent studies have revealed that Nrf2 also contributes to hepatic lipid metabolism. The deletion of Nrf2 results in the rapid onset and progression of hepatic steatosis induced by a methionine-choline-deficient or high-fat diet, suggesting the importance of the Nrf2-mediated defense system in liver metabolism.

In contrast to Nrf2, the molecular function of Nrf1 is not well defined. It has been reported that Nrf1 is anchored to the endoplasmic reticulum membrane (Literature 2); however, it is not well understood how Nrf1 activity is regulated. Nevertheless, the functional importance of Nrf1 has been demonstrated by gene targeting studies. Nrf1 knockout mice are embryonic lethal on 13.5th day due to anemia (Literature 3). Central nervous system-specific Nrf1 knockout mice show progressive motor ataxia and neurodegeneration with an accumulation of polyubiquitinated proteins in their neurons (Literature 4 and 5). The present inventor group and other groups have reported that hepatocyte-specific Nrf1 knockout mice develop hepatic steatosis (Literature 6 and 7). Recent studies suggest that Nrf1 is involved in the induction of the proteasome subunit genes (Literature 8 and 2). In addition, another study reports that degradation of Nrf1 is controlled by β-transducin repeat-containing protein (β-TrCP) and Hrd1 (Literature 9). However, the relevant downstream target genes related to hepatic steatosis in Nrf1 mutant mice still remain poorly understood.

### CITATION LIST

Literature 1: Itoh, K. et al., TBiochem. Biophys. Res. Commun. 236: 313-322. (1997)
Literature 2: Steffen, J. et al., Mol. Cell. 40: 147-158. (2010)
Literature 3: Chan, J. et al., EMBO J. 17: 1779-1787. (1998)
Literature 4: Kobayashi, A. et al., Genes Cells 16: 692-703. (2011)
Literature 5: Lee, C. S. et al., Proc. Natl. Acad. Sci. USA. 108: 8408-8413. (2011)
Literature 6: Xu, Z. R. et al., Proc. Natl. Acad. Sci. USA. 102: 4120-4125. (2005)
Literature 7: Ohtsuji, M. et al., J. Biol. Chem. 283: 33554-33562. (2008)
Literature 8: Radhakrishnan, S. K. et al., Mol. Cell. 38: 17-28. (2010)
Literature 9: Tsuchiya, Y. et al., Mol. Cell. Biol. 31: 4500-4512. (2011)

### DISCLOSURE OF INVENTION

Compounds regulating Nrf1 activity could be candidate compounds for prophylaxis/therapeutic agents for disorders such as hepatic steatosis. Under the circumstances, methods for screening for compounds regulating Nrf1 activity have been desired. Particularly, in vitro measurement systems for screening for Nrf1-specific compounds, reflecting in vivo Nrf1 function, have not yet been known.

The present inventors have conducted intensive studies with a view toward solving the above problems. Specifically, to delineate the contribution of Nrf1 to liver metabolism, they investigated the global gene expression patterns in Nrf1-deficient livers, and found that expression of genes related to lipid and amino acid metabolism and mitochondrial respiratory function is dysregulated in Nrf1-deficient liver. This suggested that the lack of Nrf1 function leads to dysregulation of several metabolic pathways. Interestingly, these pathways were not largely changed in the livers of Nrf2-deficient mice and Keap1 knockdown (KD) mice, suggesting distinct roles of Nrf1 and Nrf2 in liver metabolism. In addition, the mRNA levels of metabolic transcriptional coactivators, Lipin1 and PGC-1β decreased in Nrf1-deficient liver. Chromatin immunoprecipitation analyses revealed that Nrf1 and its heterodimeric partner, MafG, are recruited to the AREs of Lipin1 and PGC-1β. In vitro experiments confirmed that Nrf1 binds to AREs of Lipin1 and PGC-1β genes and activates transcription. These data suggest that Nrf1 is a transcription factor directly controlling expression of Lipin1 and PGC-1β. Based on these, the present inventors found that screening for compounds regulating Nrf1 activity can be performed using expression levels and/or activities of Lipin1 and PGC-1β as an index and accomplished the present invention.

More specifically, the present invention provides a method for screening for a compound regulating Nrf1 activity, a method for screening for a compound enhancing Nrf1 stability, a method for screening for a compound promoting lipid degradation and the like, as follows.

[1] A method for screening a test compound for a compound regulating Nrf1 activity, including using Nrf1 and Lipin1 and/or PGC-1β.

[2] The method according to the above [1], including (i) measuring an expression level or activity of Lipin1 and/or PGC-1β in a cell expressing Nrf1 and Lipin1 and/or PGC-1β in a case where the test compound is contacted with the cell; (ii) measuring an expression level or activity of Lipin1 and/or PGC-1β in the cell expressing Nrf1 and Lipin1 and/or PGC-1β in a case where the test compound is not contacted with the cell; and (iii) comparing the expression levels or activities of Lipin1 and/or PGC-1β in the cases (i) and (ii).

[3] The method according to the above [2], in which the test compound is selected as a compound enhancing Nrf1 activity if the expression level or activity of Lipin1 and/or PGC-1β in the case (i) is higher than the expression level or activity of Lipin1 and/or PGC-1β in the case (ii).

[4] The method according to the above [1], including (i) measuring an expression level or activity of a marker gene in a cell, into which a recombinant vector having the marker gene ligated downstream of an ARE region in Lipin1 and/or PGC-1β intron is introduced, in a case where the test compound is contacted with the cell; (ii) measuring an expression level or activity of the marker gene in the cell in a case where the test compound is not contacted with the cell; and (iii) comparing expression levels or activities of the marker gene in the cases (i) and (ii).

[5] The method according to the above [4], in which the test compound is selected as a compound enhancing Nrf1 activity if the expression level or activity of the marker gene in the case (i) is higher than the expression level or activity of the marker gene in the case (ii).

[6] A method for screening a test compound for a compound enhancing Nrf1 stability, including using a cell expressing an Nrf1-marker fusion protein.

[7] The method according to the above [6], including (i) measuring an amount of the Nrf1-marker fusion protein in a cell expressing the Nrf1-marker fusion protein in a case where the test compound is contacted with the cell; (ii) measuring an amount of the Nrf1-marker fusion protein in the cell expressing the Nrf1-marker fusion protein in a case where the test compound is not contacted with the cell; and (iii) comparing the amounts of the Nrf1-marker fusion protein in the cases (i) and (ii).

[8] The method according to the above [7], in which the test compound is selected as a compound enhancing Nrf1 stability if the amount of the Nrf1-marker fusion protein in the case (i) is higher than the amount of the Nrf1-marker fusion protein in the case (ii).

[9] The method according to any one of the above [1] to [5], in which a compound enhancing Nrf1 stability obtained by screening using the cell expressing Nrf1-marker fusion protein is used as the test compound.

[10] A method for screening for a compound promoting lipid degradation, including selecting a compound promoting lipid degradation from the compound obtained by the method according to any one of the above [1] to [9].

The present invention provides a method for screening a test compound for a compound regulating Nrf1 activity, including using Nrf1 and Lipin1 and/or PGC-1β. Since Lipin1 and/or PGC-1β are genes the expression of which is directly controlled by Nrf1, use of these successfully provides an Nrf1-specific screening system reflecting in vivo Nrf1 function.

### BRIEF DESCRIPTION OF DRAWINGS

FIG. 1 shows the results of microarray analyses for identifying the enrichment of genes associated with various metabolic pathways. (A) Table of analysis results of Reactome pathways with genes that are downregulated (left panel) or upregulated (right panel) in Nrf1 CKO mouse livers. (B) GSEA histograms for the gene sets involved in “fatty acid metabolism”, “oxidative phosphorylation” and “cell cycle regulation”. The nominal p-values are all less than 0.001, and the normalized enrichment scores are 1.28, 1.52 and −1.70, respectively. At the bottom of each plot, the spectrum of gene expression observed in the microarray is shown. Being on the left side in the figures indicates reduced expression and being on the right side indicates increased expression in the Nrf1 CKO mice. (C) The expression of representative genes in the fatty acid metabolism pathway (Acox1, Hadh, Akr1d1, Hmgcs2 and Fads1) was examined by qPCR. (D) The expression of genes related to methionine metabolism (Mat1a, Ahcy, Gnmt and Nnmt) was examined by qPCR. RNA samples were prepared from the livers of Nrf1^{dN/+} (Control) and Nrf1^{dN/-}: Alb-Cre (CKO) mice at 6 weeks of age (C, D). The data represent the mean±SD, n=3, with p values from Student's unpaired t-test denoted as follows: *p<0.05, **p<0.01. The expression of each gene in the control mouse was set to 1.

FIG. 2 shows that microarray analyses reveal differential gene expression profiles in the livers of Nrf1 CKO, Nrf2 knockout, and Keap1 KD mice. (A) The genotypes used for microarray analyses are shown. (B) The heat map of differentially regulated genes (≧1.5-fold change at p≦0.05 (t-test)) in Nrf1 CKO mice and the expression data for the Nrf2 knockout and Keap1 KD mice are shown. (C) The Venn diagram, indicating the degree of overlap among the genes decreased in the Nrf1 CKO mice, the genes decreased in Nrf2 knockout mice, and the genes increased in Keap1 KD mice. (D to H) The heat map of the genes that were differentially expressed in the Nrf1 CKO mice that belong to the categories of lipid metabolism (D), amino acid metabolism (E), proteasome subunits (F), mitochondrial respiratory chain (G), and typical Nrf2 target genes (H), is shown together with the expression data for the Nrf2 knockout and Keap1 KD mice. The colors of the heat map reflect the log(2)-fold-change values relative to the expression of each gene in the control mice. The gene symbols used here are consistent with those used in the Mouse Genome Informatics (MGI) database.

FIG. 3 shows expression of transcriptional regulators associated with hepatic lipid metabolism in Nrf1-deficient livers. The mRNA expression was examined by qPCR in the livers of Nrf1^{dN/+} (Control) and Nrf1^{dN/-}: Alb-Cre (CKO) mice at 4 and 6 weeks of age. The data represent the mean±SD, n=3, with p values from Student's unpaired t-test, denoted as follows: *p<0.05, **p<0.01. The expression of each gene in the control mouse was set to 1.

FIG. 4 shows that Nrf1 knockdown results in the reduced expression of PPARα, Lipin1 and PGC-1β. Hepa1 cells stably expressing shRNA targeting Nrf1 or a non-silencing control gene were used. The mRNA expression in five independent clones was detected by qPCR. The data represent the mean±SD, n=3, **p<0.01. Expression of each gene in the control mouse was set to 1.

FIG. 5 shows that Nrf1 and MafG are recruited to the AREs of Lipin1 and PGG-1β. (A) Alignment of MARE and ARE found in the regulatory regions of PPARα, Lipin1, and PGC-1β. The chromosome numbers and positions on the chromosome are indicated according to Mouse Genome Database (NCBI37/mm9). (B, C) ChIP assays were performed with chromatin extracts from Hepa1 cells using normal IgG (solid bars) and anti-Nrf1 or anti-MafG antibodies (open bars) and analysis by qPCR was performed using primers flanking the AREs in the promoter (P) and 5′URT (U) of PPARα, the promoter and intron (I) of Lipin1 and the intron of PGC-1β. The data represent the mean±SD, n=3. The inset represents a magnified graph of the ChIP analysis for PPARα and Lipin1 genes.

FIG. 6 shows that Nrf1 binds to the AREs of Lipin1 and PGC-1β and activates transcription in vitro. (A to C) Hepa1 cells were cotransfected with luciferase reporter constructs containing the AREs from the Lipin1 promoter (A), Lipin1 intron (B) or PGC-1β intron (C) together with or without the Nrf1 expression vector. The average values±S.D are shown, n=3. The vertical axis indicates relative luciferase units (RLU). The firefly luciferase activity in the absence of effector plasmids is set to 1. (D) The EMSA analysis was performed with biotin-labeled probes containing the AREs from the Nqo1 promoter, Lipin1 promoter, Lipin1 intron and PGC-1β intron regions. Recombinant MafG and/or Nrf1 or Nrf2 proteins were incubated with the probes, and the protein-DNA complexes and free probes were resolved by electrophoresis. (E) The sequences of the wild-type (W) and mutant (M) oligonucleotides are shown. The mutation sites of the sequences are underlined. (F) Nrf2 and MafG proteins were incubated in combination in the presence or absence of a 200-fold molar excess of unlabeled competitor DNA. The open arrowheads indicate Nrf1/MafG heterodimer complexes, the solid arrowheads indicate Nrf2-MafG heterodimer complexes and the arrows indicate MafG homodimer complexes.

FIG. 7 shows that Nrf2 does not participate in the regulation of Lipin1 and PGC-1β. (A to B) Expression of Nqo1, Gc1c, Lipin1 and PGC-1β in Hepa1 cells treated with 100 μM DEM or DMSO (Veh) for 6 hours (A) or in livers of Keap1 KD mice (B). The mRNA expression was analyzed by qPCR. The data represent the mean±SD, n=3. (C to F) The ChIP analyses were performed with chromatin extracts from Hepa1 cells (C, E) treated with 100 μM DEM or DMSO (Veh) for 4 hours or from the livers of Keap1 KD mice (D, F) using anti-Nrf2 (C, D) or anti-MafG (E, F) antibodies (open bars). Normal IgG was used as a negative control (solid bars). The amount of immunoprecipitated DNA was analyzed by qPCR with primers flanking the ARE regions in the Nqo1 promoter, the Lipin1 promoter and the Lipin1 and PGC-1β introns. The third intron of the thromboxane synthase (Tx) gene was used as a negative control. The data represent the mean±SD, n=3.

FIG. 8 shows generation of Nrf1 CKO using the Neo-deleted (dN) allele. (A) The structures of the Nrf1 wild-type (WT), Nrf1-floxed, dN, and Nrf1-deleted (KO) alleles are shown. Neo gene was removed from Nrf1-floxed allele to generate dN allele by mating with Ayu1-Cre mice that express ubiquitous Cre recombinase. (B) The Nrf1 mRNA expression in the livers from Nrf1 CKO and control mice (3, 4, and 6 weeks of age) was examined by qPCR. (C) The serum ALT activity in the Nrf1 CKO and control mice was examined. The data represent the mean±SD, n=3, and U represents unit. (D to G) Liver sections from Nrf1^{dN/+} (Control) and Nrf1^{dN/-}: Alb-Cre (CKO) mice at 3 weeks (D, E) and 5 weeks (F, G) of age were stained with oil red 0. The scale bar corresponds to 100 μm.

FIG. 9 shows luciferase activity in Nrf1-luciferase fusion protein expression cell. (duplicate, Mean±SD)

### BEST MODE FOR CARRYING OUT THE INVENTION

Now, the present invention will be more specifically described below.

### 1. Nrf1, Lipin1 and PGG-1β

### 1.1. Nrf1

“Nrf1” (nuclear factor erythroid 2-related factor 1) is a member of the cap'n′ collar (CNC) transcription factor family. Nrf1 is regarded as an important regulator for various biological processes including metabolism. Nrf1 migrates from cytoplasm to nucleus, binds to ARE (Antioxidant response element) sequences through formation of heterodimers with Maf proteins, and activates a target gene. The target genes of Nrf1 are, for example, Lipin1 and PGC-1β genes.

In the present invention, Nrf1 is not particularly limited; however, for example, human-derived Nrf1 and mouse-derived Nrf1 are mentioned. The gene and amino acid sequences of human Nrf1 are registered in the GenBank as Accession No. NM_{—}003204 (gene) (SEQ ID NO: 1) and Accession No. NP_{—}003195 (protein) (SEQ ID NO: 2), respectively. The gene and amino acid sequences of mouse Nrf1 are registered in the GenBank as Accession No. NM_{—}008686 (gene) (SEQ ID NO: 3) and Accession No. NP_{—}032712 (protein) (SEQ ID NO: 4), respectively.

In the specification, Nrf1 used here is a term including its mutants as long as they have substantially the same activity as Nrf1. Examples of the mutants include proteins having the Nrf1 amino acid sequence with deletion, substitution, insertion and/or addition of 1 to a plurality of amino acids (for example, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9 (1 to several), 1 to 8, 1 to 7, 1 to 6, 1 to 5, 1 to 4, 1 to 3, 1 to 2, or 1). Generally, the smaller the number of amino acids of deletion, substitution, insertion or addition, the more preferable. Two or more events of deletion, substitution, insertion and addition of amino acid residues may occur simultaneously.

As the substantially the same activity, for example, transcription enhancing activity of the Nrf1 target gene and ARE binding activity are mentioned. The “substantially the same” means that these activities are equivalent in nature (e.g., physiologically, or pharmacologically). Therefore, it is preferable to have the equivalent ARE binding activity and Nrf1 target gene transcription enhancing activity, etc. (e.g., about 0.01 to 100 fold, preferably about 0.1 to 10 fold, more preferably 0.5 to 2 fold). The quantitative factors such as degrees of these activities and the molecular weights of proteins may vary.

Note that, in the specification, “Nrf1” generally refers to Nrf1 protein but sometimes refers to Nrf1 gene depending on the context.

### 1.2. Lipin1

“Lipin1”, together with Lipin 2, Lipin3, etc., is a protein belonging to a Lipin family. Lipin1 is known to have two functions in the regulation of lipid metabolism due to a difference in intracellular localization. First, Lupin1 has phosphatidic acid phosphatase activity on endoplasmic reticulum membrane. Second, Lipin1 acts as a coactivator of PPARα/PGC-1α regulatory pathway within a nucleus and positively regulates oxidative metabolism of fatty acid.

In the present invention, Lipin1 is not particularly limited; however, for example, human-derived Lipin1 and mouse-derived Lipin1 are mentioned. The gene and amino acid sequences of human Lipin1 are registered in the GenBank as Accession No. NM_{—}145693 (gene) (SEQ ID NO: 5) and Accession No. NP_{—}663731 (protein) (SEQ ID NO: 6), respectively. The gene and amino acid sequences of mouse Lipin1 are registered in the GenBank as Accession No. NM_{—}172950 (gene) (SEQ ID NO: 7) and Accession No. NP_{—}766538 (protein) (SEQ ID NO: 8), respectively.

In the specification, Lipin1 used here is a term including its mutants as long as they have substantially the same activity as Lipin1. Examples of the mutants include proteins having the Lipin1 amino acid sequence with deletion, substitution, insertion and/or addition of 1 to a plurality of amino acids (for example, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9 (1 to several), 1 to 8, 1 to 7, 1 to 6, 1 to 5, 1 to 4, 1 to 3, 1 to 2, or 1). Generally, the smaller the number of amino acids of deletion, substitution, insertion or addition, the more preferable. Two or more events of deletion, substitution, insertion and addition of amino acid residues may occur simultaneously.

As the substantially the same activity, for example, phosphatidic acid phosphatase activity and coactivator activity of PPARα/PGC-1α regulatory pathway are mentioned. The “substantially the same” means that these activities are equivalent in nature (e.g., physiologically, or pharmacologically). Therefore, it is preferable to have the equivalent phosphatidic acid phosphatase activity and coactivator activity of PPARα/PGC-1α regulatory pathway, etc. (e.g., about 0.01 to 100 fold, preferably about 0.1 to 10 fold, more preferably 0.5 to 2 fold). The quantitative factors such as degrees of these activities and the molecular weights of proteins may vary.

Note that, in the specification, “Lipin1” generally refers to Lipin1 protein but sometimes refers to Lipin1 gene depending on the context.

### 1.3. PGC-1β

“PGC-1β” (peroxisome proliferator-activated receptor γ coactivator 1β) is a protein required for coactivating ERRα (estrogen-related receptor a) and nuclear respiratory factor 1. PGC-1β knockout mice show an altered expression of mitochondrial oxidative metabolism genes and high fat diet-induced hepatic steatosis.

In the present invention. PGC-1β is not particularly limited; however, for example, human-derived PGC-1β and mouse-derived PGC-1β are mentioned. The gene and amino acid sequences of human PGC-1β are registered in the GenBank as Accession Na. NM_{—}133263 (gene) (SEQ ID NO: 9) and Accession No. NP_{—}573570 (protein) (SEQ ID NO: 10), respectively. The gene and amino acid sequences of mouse PGC-1β are registered in the GenBank as Accession No. NM_{—}133249 (gene) (SEQ ID NO: 11) and Accession No. NP_{—}573512 (protein) (SEQ ID NO: 12), respectively.

In the specification, PGC-1β used here is a term including its mutants as long as they have substantially the same activity as PGC-1β. Examples of the mutants include proteins having the PGC-1β amino acid sequence with deletion, substitution, insertion and/or addition of 1 to a plurality of amino acids (for example, 1 to 30, 1 to 29, 1 to 28, 1 to 27, 1 to 26, 1 to 25, 1 to 24, 1 to 23, 1 to 22, 1 to 21, 1 to 20, 1 to 19, 1 to 18, 1 to 17, 1 to 16, 1 to 15, 1 to 14, 1 to 13, 1 to 12, 1 to 11, 1 to 10, 1 to 9 (1 to several), 1 to 8, 1 to 7, 1 to 6, 1 to 5, 1 to 4, 1 to 3, 1 to 2, or 1). Generally, the smaller the number of amino acids of deletion, substitution, insertion or addition, the more preferable. Two or more events of deletion, substitution, insertion and addition of amino acid residues may occur simultaneously.

As the substantially the same activity, for example, coactivation activity of ERRα and nuclear respiratory factor 1 is mentioned. The “substantially the same” means that these activities are equivalent in nature (e.g., physiologically, or pharmacologically). Therefore, it is preferable to have the equivalent coactivation activity of ERRα and nuclear respiratory factor 1, etc. (e.g., about 0.01 to 100 fold, preferably about 0.1 to 10 fold, more preferably 0.5 to 2 fold). The quantitative factors such as degrees of these activities and the molecular weights of proteins may vary.

Note that, in the specification, “PGC-1β” generally refers to PGC-1β protein but sometimes refers to PGC-1β gene depending on the context.

### 2. Screening Method

### 2.1. Method for Screening for Compounds Regulating Nrf1 Activity

The present invention provides a method for screening test compounds for compounds regulating Nrf1 activity using Nrf1 and Lipin1 and/or PGC-1β. Compounds regulating Nrf1 activity refer to compounds enhancing or inhibiting Nrf1 activity; and preferably compounds enhancing Nrf1 activity.

According to a preferable embodiment of the present invention, there is provided a method for screening for compounds regulating Nrf1 activity using the expression level or activity of Lipin1 and/or PGC-1β, as an index.

According to another preferable embodiment of the present invention, there is provided a screening method including ligating a marker gene downstream of an ARE region in Lipin1 and/or PGC-1β intron and screening for compounds regulating Nrf1 activity using the expression level or activity of the marker gene as an index.

Examples of test compounds include peptides, proteins, antibodies, non-peptide compounds, synthetic compounds, fermented products, cell extracts, plant extracts, animal tissue extracts and blood plasma. These compounds may be either novel compounds or known compounds. Test compounds may form salts. Examples of the salts of test compounds include physiologically accepted metal salts, ammonium salts, salts with organic bases, salts with inorganic acids, salts with organic acids and salts with basic or acidic amino acids. Preferable examples of the metal salts include alkaline metal salts such as sodium salts and potassium salts; alkaline earth metal salts such as calcium salts, magnesium salts, and barium salts; and aluminum salts. Preferable examples of the salts with organic bases include salts with trimethylamine, triethylamine, pyridine, picoline, 2,6-lutidine, ethanolamine, diethanolamine, triethanolamine, cyclohexylamine, dicyclohexylamine and N,N′-dibenzylethylenediamine. Preferable examples of the salts with inorganic acids include salts with hydrochloric acid, hydrobromic acid, nitric acid, sulfuric acid and phosphoric acid. Preferable examples of the salts with organic acids include salts with formic acid, acetic acid, trifluoroacetic acid, propionic acid, phthalic acid, fumaric acid, oxalic acid, tartaric acid, maleic acid, citric acid, succinic acid, malic acid, methanesulfonic acid, benzoic acid, benzenesulfonic acid and p-toluenesulfonic acid. Preferable examples of the salts with basic amino acids include salts with arginine, lysine and ornithine. Preferable examples of the salts with acidic amino acids include salts with aspartic acid and glutamic acid.

A preferable embodiment of the screening method of the present invention, for example, includes (i) measuring the expression level or activity of Lipin1 and/or PGC-1β in cells expressing Nrf1 and Lipin1 and/or PGC-1β in a case where the test compounds are contacted with the cells; (ii) measuring the expression level or activity of Lipin1 and/or PGC-1β in the cells expressing Nrf1 and Lipin1 and/or PGC-1β in a case where the test compounds are not contacted with the cells; and (iii) comparing expression levels or activities of Lipin1 and/or PGC-1β in the cases (i) and (ii).

In this method, first, test compounds are contacted with cells expressing Nrf1 and Lipin1 and/or PGC-1β. Origins of the “cells” that are used are not particularly limited. Examples of the cells include cells derived from humans and mice, and preferably cells derived from humans. Examples of the “cells expressing Nrf1 and Lipin1 and/or PGC-1β” used in the screening method of the present invention include hepatocytes and hepatoma-derived cell lines. The “cells expressing Nrf1 and Lipin1 and/or PGC-1β” used in the screening method of the present invention can be also prepared in general genetic engineering techniques.

Then, the expression level or activity of Lipin1 and/or PGC-1β is measured. Specifically, for example, in the above cases (i) and (ii), the above cells are cultured and expression level or activity of Lipin1 and/or PGC-1β is measured in each of the cases. In principle, it is preferred to measure the expression level. As an alternative index of the expression level, activity may be measured. The intensity of the activity is proportional to the expression level. The expression level or activity of Lipin1 and/or PGC-1β can be measured by known methods or methods corresponding to the known methods. For example, the expression level of Lipin1 and/or PGC-1β can be detected using, e.g., microarrays, PCR and antibodies.

Subsequently, compounds enhancing (or inhibiting) the expression level or activity of Lipin1 and/or PGC-1β are selected through comparison with the case (a control) where test compounds are not contacted with the cells. For example, test compounds are selected as test compounds enhancing the expression level or activity of Lipin1 and/or PGC-1β if the expression level or activity of Lipin1 and/or PGC-1β in the case (i) is higher than the expression level or activity of Lipin1 and/or PGC-1β in the case (ii); particularly, higher by 10% or more, 20% or more, 30% or more, 40% or more, 50% or more. The test compounds enhancing the expression level or activity of Lipin1 and/or PGC-1β selected in this manner could be compounds enhancing Nrf1 activity. Similarly, compounds inhibiting Nrf1 activity may be selected using reduction in expression level or activity of Lipin1 and/or PGC-1β as an index.

In another preferable embodiment of the screening method of the present invention, a marker gene is ligated downstream of an ARE region in Lipin1 and/or PGC-1β intron and compounds regulating Nrf1 activity are screened using the activity of the marker gene as an index.

This method includes, for example, (i) measuring the expression level or activity of a marker gene in cells, into which a recombinant vector having the marker gene ligated downstream of an ARE region in Lipin1 and/or PGC-1β intron is introduced, in a case where the test compounds are contacted with the cells; (ii) measuring the expression level or activity of the marker gene in the cells in a case where the test compounds are not contacted with the cells; and (iii) comparing the expression levels or activities of the marker gene in the cases (i) and (ii).

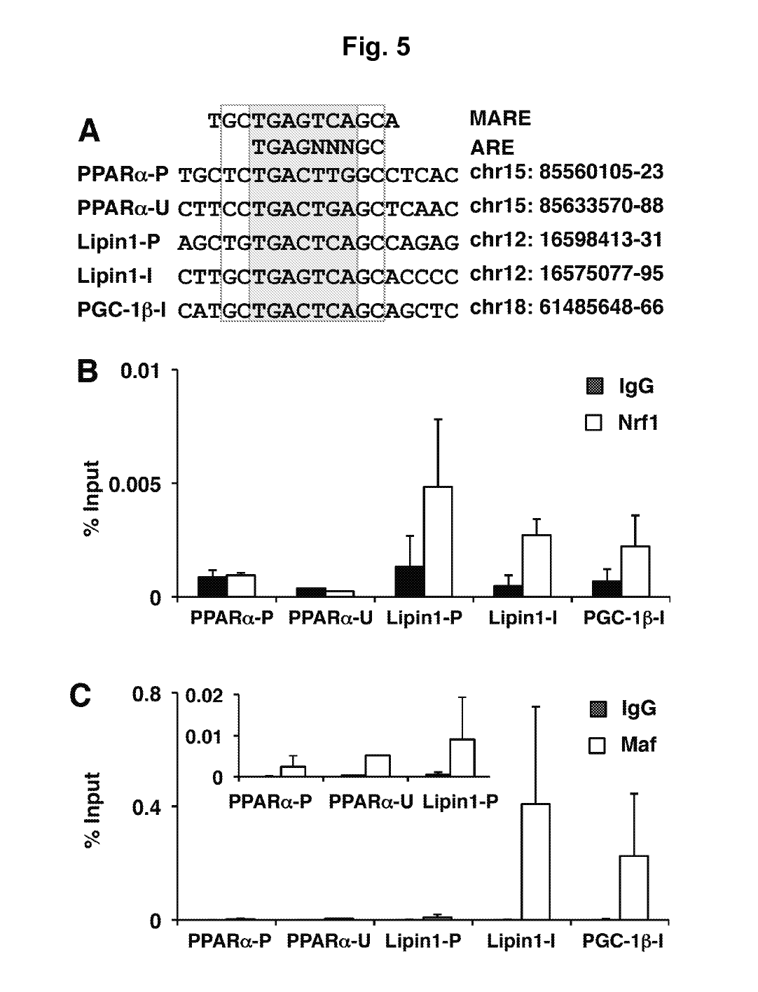

The ARE region in Lipin1 and/or PGC-1β intron to be used in this method contains the nucleotide sequence shown in FIG. 5A, preferably contains the nucleotide sequence represented by SEQ ID NO: 13 (CTTGCTGAGTCAGCACCCC) or SEQ ID NO: 14 (CATGCTGACTCAGCAGCTC). As a marker gene, for example, genes with luminescent, fluorescent and chromogenic markers such as luciferase, GFP and galactosidase can be used. A recombinant vector in which a marker gene is ligated downstream of an ARE region in a Lipin1 and/or PGC-1β intron and cells to which the recombinant vector is introduced can be prepared by general genetic engineering techniques. The cells to be used are not particularly limited; however, for example, mammal-derived cells are preferable and hepatocytes and hepatoma-derived cell lines are favorable. Furthermore, a recombinant vector expressing Nrf1 may be introduced into the cells. Whether endogenous Nrf1 is used or Nrf1 is exogenously expressed can be appropriately selected depending upon the cell strain and sensitivity of the measurement system to be used.

Then, the expression level or activity of the marker gene is measured. The activity of the target gene refers to the activity of the marker protein (luciferase, etc.) resulting from expression of marker gene. Since the intensity of the target gene activity is proportional to the expression level of the marker gene, the expression level can be calculated by measuring the activity. Specifically, for example, in the above cases of (i) and (ii), the above cells are cultured and expression level or activity of the marker gene in each of the cases is measured. The expression level or activity of the marker gene can be measured by known methods or methods corresponding to the known methods.

Subsequently, compounds enhancing (or inhibiting) the expression level or activity of the marker gene are selected though comparison with the case (control) where test compounds are not contacted with the cells. For example, test compounds are selected as test compounds enhancing Nrf1 activity if the expression level or activity of the marker gene in the case (i) is higher than the expression level or activity of the marker gene in the case (ii); particularly, higher by 10% or more, 20% or more, 30% or more, 40% or more, 50% or more. Similarly, compounds inhibiting Nrf1 activity may be selected using reduction in expression level or activity of the marker gene as an index.

### 2.2. Method for Screening for Compounds Enhancing Nrf1 Stability

The present invention further provides a method for screening test compounds for compounds enhancing Nrf1 stability, including using cells expressing Nrf1-marker fusion protein (hereinafter sometimes referred to as a “primary screening method”). Compounds enhancing Nrf1 stability obtained by the primary screening method of the present invention can be used as test compounds in a method for screening for compounds regulating Nrf1 activity (hereinafter sometimes referred to as a “secondary screening method”). Through the use of the primary screening method of the present invention, Nrf1 specific compounds can be obtained by the following secondary screening method.

The test compounds used in the primary screening method of the present invention are the same as mentioned above. Nrf1 is degraded by a proteasome under static conditions. Upon activation stimuli, Nrf1 protein gets out of the degradation system and stabilized. In this way, Nrf1 is considered to activate transcription of a target gene. Even if Nrf1-marker fusion protein is expressed in cells, the protein is degraded under static conditions. When a compound enhancing Nrf1 stability is contacted herein, degradation of Nrf1-marker fusion protein is suppressed and the amount of detectable marker protein increases.

“Nrf1-marker fusion protein” refers to a fusion protein of Nrf1 and a marker protein (for example, luminescent, fluorescent, chromogenic protein such as luciferase, GFP and galactosidase).

More specifically, in a preferable embodiment of the primary screening method of the present invention, screening for compounds enhancing Nrf1 stability is performed using the amount of Nrf1-marker fusion protein as an index, for example, by (i) measuring the amount of Nrf1-marker fusion protein in cells expressing Nrf1-marker fusion protein in a case where the test compounds are contacted with the cells; (ii) measuring the amount of Nrf1-marker fusion protein in the cells expressing Nrf1-marker fusion protein in a case where the test compounds are not contacted with the cells; and (iii) comparing the amounts of Nrf1-marker fusion protein in the cases (i) and (ii).

In this method, first, test compounds are contacted with cells expressing Nrf1-marker fusion protein. The cells that are used here are not particularly limited; however, mammal-derived cells are preferable and hepatocytes and hepatoma-derived cell lines are favorable. The “Nrf1-marker fusion protein cells” to be used in the screening method of the present invention can be prepared by a general genetic engineering techniques.

Then, the amount of Nrf1-marker fusion protein is measured. Specifically, for example, in the above (i) and (ii) cases, the above cells are cultured and the amount of Nrf1-marker fusion protein is measured in each of the cases. The amount of Nrf1-marker fusion protein can be measured by known methods or the methods corresponding to the known methods. Specifically, the activity of a marker protein of a fusion protein (for example, luminescent, fluorescent, chromogenic proteins such as luciferase, GFP and galactosidase) is measured.

Subsequently, compounds increasing the amount of Nrf1-marker fusion protein are selected through comparison with the case (control) where test compounds are not contacted with the cells. For example, test compounds can be selected as compounds enhancing Nrf1 stability if the amount of Nrf1-marker fusion protein in the case (i) is higher than the amount of Nrf1-marker fusion protein in the case (ii); particularly higher by 10% or more, 20% or more, 30% or more, 40% or more, 50% or more.

2.3. Screening Method by Combination of Nrf1KO Cells and/or Nrf2KO Cells

The present invention further provides a method for screening test compounds for compounds regulating Nrf1 activity, including using Nrf1KO cells and/or Nrf2KO cells.

Nrf1KO (knockout) cells do not have endogenous Nrf1. If the compounds obtained by the primary screening method and/or secondary screening method are subjected to the same primary screening method and/or secondary screening method using Nrf1 KO cells, it is possible to determine whether the function of the compounds is specific to Nrf1 or not. As an example of the Nrf1KO cells, MEF (mouse embryo fibroblasts) derived from Nrf1KO mice (Literature 3) can be used. Furthermore, hepatocytes derived from hepatocyte specific Nrf1 KO mice (Literature 7) may be used.

Nrf2KO (knockout) cells do not have endogenous Nrf2. If the compounds obtained by the primary screening method and/or secondary screening method are subjected to the same primary screening method and/or secondary screening method using Nrf2KO cells, it is possible to determine whether or not the compounds act on not only Nrf1 but also Nrf2. As Nrf2KO cells, for example, MEF and hepatocytes derived from Nrf2KO mice (Literature 1) can be used.

Through combination use of Nrf1 KO cells and Nrf2KO cells, compounds that act not on Nrf2 but specifically on Nrf1 can be identified.

### 2.4. Method for Screening for Compounds Promoting Lipid Degradation

Compounds enhancing Nrf1 activity and obtained by the above screening method are candidate compounds enhancing lipid degradation.

Then, the candidate compounds selected are administered to laboratory animals (e.g., mice, rats) to confirm the effect of them in promoting lipid degradation. For example, the compounds are administered to model animals known to develop hepatic steatosis (e.g., db/db mice, C57BL/6N-NASH mice, FLS mice, Zucker-fa/fa rats) to confirm a lipid degradation promoting effect.

The compounds promoting lipid degradation can be used, for example, as prophylaxis/therapeutic agents, for hepatic disorders (e.g., hepatic steatosis, non-alcoholic steatohepatitis (NASH), cirrhosis and hepatoma) and hyperlipidemia, etc.

Note that all literatures and publications described in the specification are incorporated herein in their entirety regardless of their objects by reference. Furthermore, the specification incorporates by reference disclosure in the claims, specification and drawings of Japanese Patent Application No. 2012-011833 (filed Jan. 24, 2012), based on which the priority of the present application is claimed for.

The object, features, advantages and ideas of the present invention are apparent to those skilled in the art by the description of the specification. The present invention can be easily carried out by those skilled in the art based on the description of the specification. The Best Mode for Carrying out the Invention and detailed Examples illustrate preferred embodiments of the present invention and are described just for illustrating or explaining the invention. Thus, the present invention is not limited to these. The present invention can be modified in various ways based on the description of the specification within the intention and scope of the present invention disclosed herein, as is apparent to those skilled in the art.

### Example 1

### 1.1 Materials and Methods

### 1.1.1 DNA Constructs

For the reporter analysis, the following sequences containing the AREs: Lipin1 promoter (5′-ACG CTC CTG CCG CTG AGC TGT GAC TCA GCC AGA GAA CTG AG-3′; SEQ ID NO: 15), Lipin1 intron (5′-CAC ACC CTG CCC AGA GGC ACA CTT GCT GAG TCA GCA CCC CGG-3′; SEQ ID NO: 16), and PGC-1β intron (5′-TTG ATA GTG AGG GGA ACA TGC TGA CTC AGC AGC TCC GAA TAA-3′; SEQ ID NO: 17) were flanked by MluI and NheI sites and cloned into pRBGP3 vector (Igarashi et al., Nature 367: 568-572. (1994)) to generate Lipin1 promoter ARE-Luc, Lipin1 intron ARE-Luc, and PGC-1β intron ARE-Luc, respectively.

The short hairpin RNA (shRNA) expression construct targeting Nrf1 was based on a 19-mer sequence (5′-GGG ATT CGG TGA AGA TTT G-3′; SEQ ID NO: 18) present in the coding region of both human and mouse Nrf1 genes, followed by a complementary 19-nucleotide sequence, which was separated by a 9-nucleotide sequence (TTCAAGAGA) and cloned into the BglII and HindIII sites of the pSUPER.retro.puro vector (Oligoengine).

To generate constructs expressing carboxyl terminal half of Nrf1, the mouse Nrf1 cDNA (G341-K741) was amplified by PCR using 3×FLAG-Nrf1 as a template with the following primers: forward (5′-AGC CAT ATG GGC TGC AGT CAG GAC TTC TCC-3′: SEQ ID NO: 19) and reverse (5′-ATC CTC GAG TCA CTT CCT CCG GTC CTT TGG-3′: SEQ ID NO: 20) primers. The PCR products were digested with NdeI and XhoI and cloned into the pET-15b vector (Novagen) to generate 6×His-Nrf1CT.

### 1.1.2 Mouse

Nrf1 conditional and knockout alleles (Literature 7), Nrf2 knockout allele (Literature 1) and Keap1 KD allele (Taguchi et al., Mol. Cell. Biol. 30: 3016-3026. (2010)) used here were known in the art. The albumin (Alb)-Cre transgenic mice were purchased from Jackson Laboratories (Bar Harbor, Me.). The mice were given water and rodent chow ad libitum and kept under specific-pathogen-free conditions. All mice were handled according to the regulations of the Standards for Human Care and Use of Laboratory Animals of Tohoku University and Guidelines for Proper Conduct of Animal Experiments of the Ministry of Education, Culture, Sports, Science, and Technology of Japan.

### 1.1.3 Histological and Biochemical Analyses

To visualize the hepatic lipid content, the livers were embedded in OCT compound (Tissue Tek). The frozen sections were stained with oil Red O (Muto Pure Chemicals) and counterstained with hematoxylin. The plasma alanine aminotransferase (ALT) activity was measured by a method known in the art (Literature 7).

1.1.4 RNA Purification and Quantitative PCR Analyses (qPCR)

Total RNA was extracted using the ISOGEN RNA extract kit (Nippon Gene) and reverse-transcribed to cDNA using Super-script III (Invitrogen). The qPCR was performed with PCR master Mix using TaqMan probe or SYBR Green and the ABI 7300 system (Applied Biosystems). The primers and probes for the NAD(P)H: quinone oxidoreductase (Nqo1), glutamate-cysteine ligase catalytic subunit (Gcic) and thioredoxin reductase 1 (Txnrd1) detection used here were those known in the art (Katsuoka et al., Mol. Cell. Biol. 25: 8044-8051. (2005)). The expression levels were normalized to those of hypoxanthine-guanine phosphoribosyltransferase (HPRT).

### 1.1.5 Microarray Analyses and Data Mining

Three independent RNA samples obtained from each genotype of female mice were used for the microarray analyses. The Agilent 4×44K Whole-Mouse Genome Oligo Microarray slides were hybridized, washed, and scanned on an Agilent Microarray Scanner according to the Agilent protocol. The expression data were subjected to statistical analysis using GeneSpring software (Silicon Genetics, Redwood City, Calif., USA). The pathway analysis was conducted using the Reactome pathway enrichment tool (http://www.reactome.org). The gene set analysis was performed using the gene set enrichment analysis (GSEA) methods as default parameters (http://www.broadinstitute.org/gsea). Cluster 3.0 software was used for clustering, and the results were visualized using JAVA Treeview (http://jtreeview.sourceforge.net/).

### 1.1.6 ChIP Assay

The ChIP assay was performed in accordance with a method known in the art (Shang, et al. Cell 103: 843-852. (2000)). Briefly, liver tissues or liver cells were fixed with 1% formaldehyde at room temperature for 5 min and subsequently quenched with 0.125 M glycine. The fixed samples were lysed and sonicated. The antibody incubations were performed overnight at 4° C. The cross-linking was reversed at 65° C. for 2 hours. The purified DNA was analyzed by qPCR. The antibodies used here were anti-Nrf1 (Santa Cruz; sc-13031), anti-Nrf2 (Santa Cruz; sc-13032), and anti-MafG and normal rabbit IgG (Santa Cruz; sc-2027). The values obtained from the immunoprecipitated samples were normalized to the input DNA.

### 1.1.7 Cell Culture and Generation of Stable Cell Lines

The mouse hepatoma cell line Hepa1c1c7 (Hepa1) was cultured in Dulbecco's modified Eagle's medium (Wako) supplemented with 10% fetal bovine serum and 1% penicillin-streptomycin (Gibco). To establish the Nrf1 knockdown cell lines, the Nrf1 shRNA plasmid was transfected into Hepa1 cells using Lipofectamine-2000 transfection reagent (Invitrogen). Stable transformants were selected by 2 μg/ml puromycin, and several clonal cell lines were established. To induce Nrf1 activity, the Hepa1 cells were treated with 100 μM diethylmaleate (DEM) or DMSO for 4 or 6 hours.

### 1.1.8 Luciferase Assay

The Hepa1 cells were seeded at a density of 2×10⁵ cells per well in a 24-well plate. The previously described pRBGP3 luciferase vectors were cotransfected with or without the Nrf1 expression vector and pRL-TK (Promega) using Lipofectamine 2000 (Invitrogen). Twenty four hours after transfection, the luciferase activities were measured using a luminometer (Berthold). The firefly luciferase activity was normalized to the renilla luciferase activity. All samples were prepared in triplicate.

### 1.1.9 Electrophoretic Mobility Shift Assay (EMSA)

EMSA was performed in accordance with a method known in the art. Briefly, the expression vectors 6×His-Nrf1CT, 6×His-Nrf2CT and 6×His-MafG1-123 were transformed into Escherichia coli BL21 Codon Plus (DE3)-RIL (Stratagene), and the induced proteins were purified using Ni-NTA agarose (Qiagen). The binding reaction was performed using a 5′ biotin-labeled DNA probe and the purified proteins in 10 μl of binding buffer. In the competition assays, a 200-fold excess of unlabeled annealed DNA was added. The reaction mixtures were incubated at room temperature for 20 min and loaded onto 5% TBE polyacrylamide gels, transferred to a Zeta Probe nylon membrane (Bio-Rad) and visualized using the LightShift EMSA Kit (Thermo Fisher Scientific).

### 1.2 Results

### 1.2.1 Loss of Nrf1 Leads to Impairment of Various Hepatocellular Functions

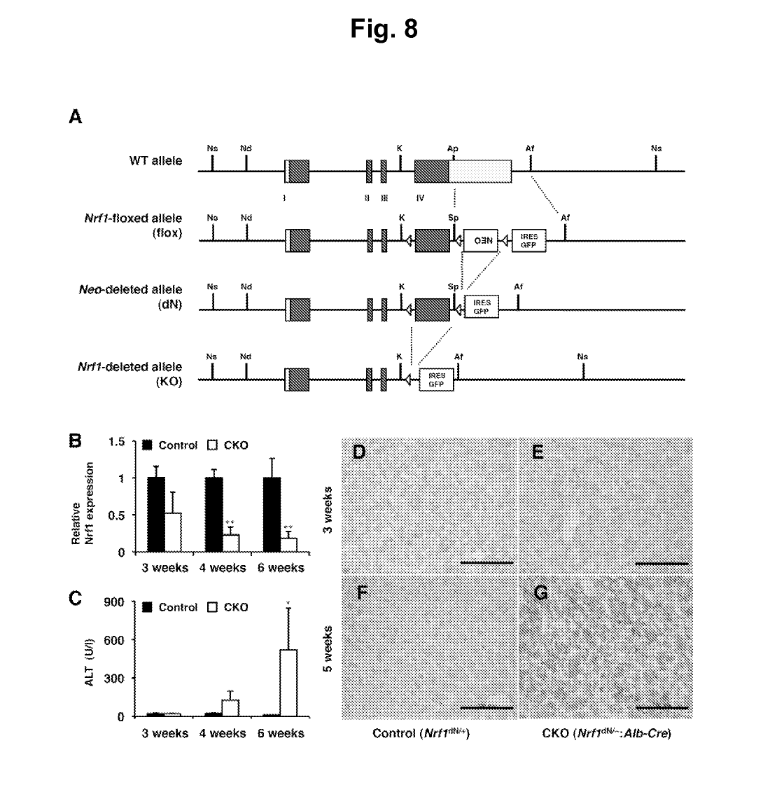

To delineate the contribution of Nrf1 to hepatic metabolism, the present inventors investigated the global gene expression patterns in Nrf1-deficient livers. For this purpose, mice harboring the foxed allele of Nrf1 were used (Literature 7). For this Example, the neomycin resistance (Neo) gene was previously removed from the Nrf1 floxed allele to allow efficient deletion of the Nrf1 gene (see details in FIG. 8A). The resulting Nrf1 Neo-deleted (dN) allele was used in combination with Alb-Cre transgenic mice to knockout Nrf1 expression specifically in hepatocytes. The hepatocyte-specific Nrf1 conditional knockout mice (Nrf1 CKO; Nrf1^{dN/-}: Alb-Cre) showed a significant increase in plasma ALT levels at 4 weeks of age, in accordance with the progressive loss of Nrf1 expression (FIGS. 8B and C). While no obvious lipid deposits were found in the livers of Nrf1 CKO mice at 3 weeks of age (FIGS. 8D and E), increased lipid accumulation was observed in the Nrf1-deficient livers at 5 weeks of age (FIGS. 8F and G). These results showed that the Alb-Cre-mediated excision of Nrf1 immediately results in impaired lipid metabolism.

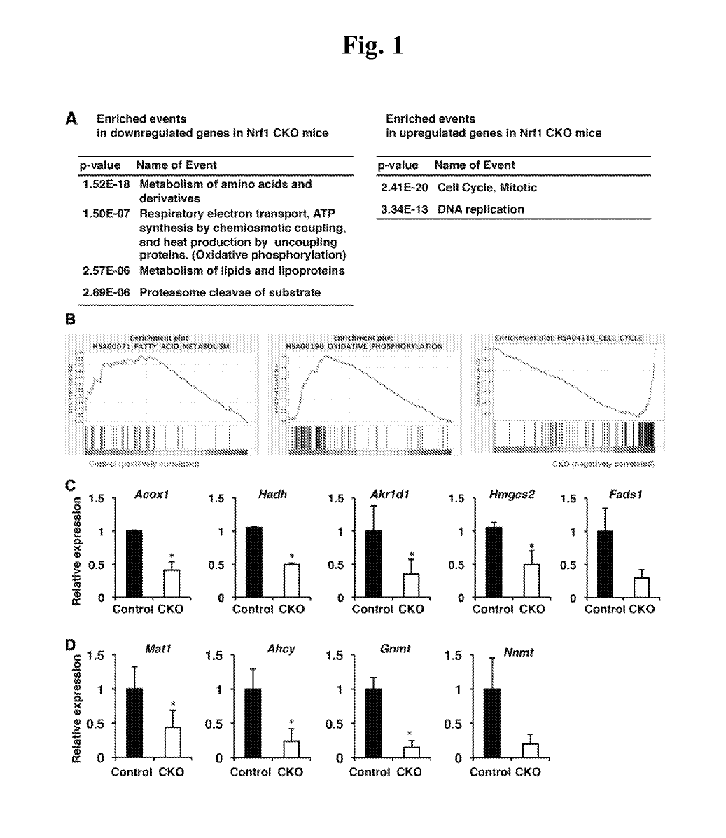

The present inventors then performed transcriptional profiling of livers in Nrf1. CKO mice and control mice (Nrf1^{dN/+}) at 6 weeks of age using microarray analysis. To minimize differences in weeks of age and time of sacrifice, which could affect metabolism, all animals were sacrificed at Zeitgeber time 9 (3 hours before lights off). Using the statistical criteria of ≧1.5-fold change with p≦0.05 (t-test), the present inventors identified approximately 1500 upregulated and 1700 downregulated genes in the Nrf1. CKO mice as compared with the control mice. The differentially expressed genes were mapped to known pathways using the Reactome database. Consistent with findings of previous studies (Literature 2 and 8), the expression levels of the proteasome subunit genes decreased in the Nrf1 CKO mice (FIG. 1A). This suggests that Nrf1 is actually functional in the liver under normal conditions. In addition, the genes involved in lipid and amino acid metabolism and the mitochondrial respiratory chain occupy a large share in the downregulated gene sets (FIG. 1A). However, the genes involved in the cell cycle and DNA replication occupy a large share in the upregulated gene sets (FIG. 1A). This suggests that cell cycle regulation is aberrant in the livers of Nrf1-deficient mice. To further verify these observations, the whole cell expression data were subjected to GSEA. Also in this assay, the gene sets related to lipid metabolism and mitochondrial respiratory chain and a gene set related to cell cycle were enriched among the downregulated and upregulated genes in the Nrf1 CKO mice, respectively (FIG. 1B).

The present inventors next focused on the expression of the PPARα target genes involved in fatty acid metabolism (FIG. 1C) because the dysregulation of these genes could lead to hepatic steatosis. The qPCR results confirmed that the expression of PPARα target genes, such as acyl-coenzyme A oxidase 1, palmitoyl (Acox1), hydroxyacyl-coenzyme A dehydrogenase (Hadh), ldo-keto reductase family 1, member D1 (Akr1d1), 3-hydroxy-3-methylglutaryl-Coenzyme A synthase 2 (Hmgcs2) and fatty acid desaturase 1 (Fads1), decreased in Nrf1 CKO mice (FIG. 1C). The present inventors also noticed that several genes related to methionine metabolism, such as methionine adenosyltransferase I, alpha (Mat1α), S-adenosylhomocysteine hydrolase (Ahcy), glycine N-methyltransferase (Gnmt) and nicotinamide N-methyltransferase (Nnmt), were also downregulated in the livers of the Nrf1-deficient mice (FIG. 1D). Taken these results in combination, it is clearly shown that the loss of Nrf1 leads to an impairment of various hepatocellular functions including lipid and amino acid metabolism.

### 1.2.2 The Gene Sets Regulated by Nrf1 are not Significantly Affected by the Loss and Gain of Nrf2 Function

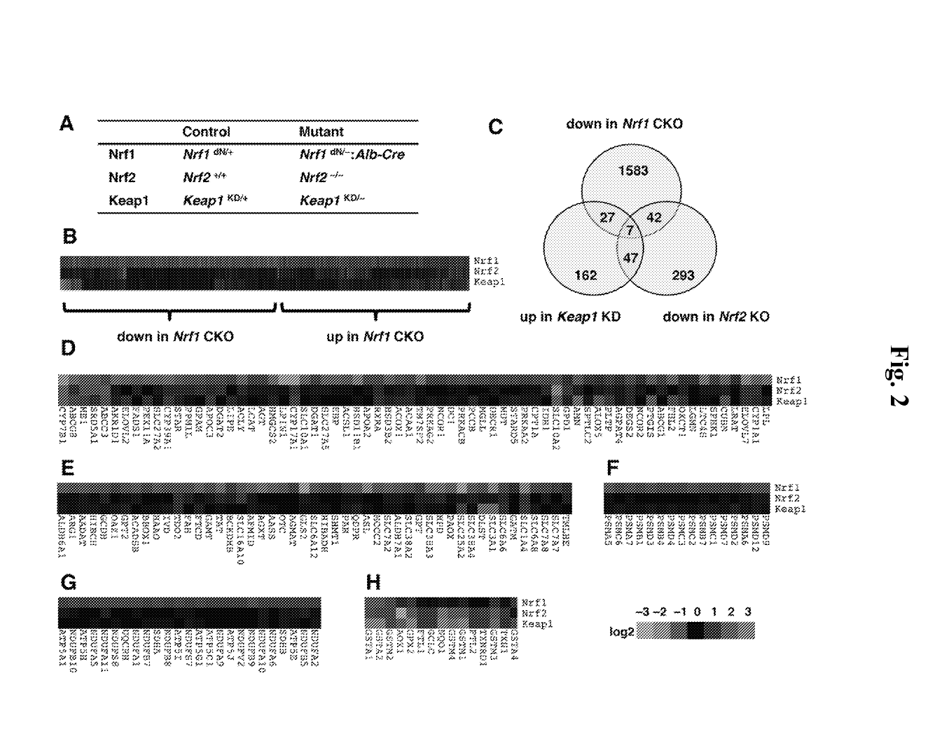

It is reported that Nrf1 and Nrf2 share functional properties; therefore, it might be possible that these genes also regulate the same gene sets as well. To test this possibility, the present inventors examined the gene expression profiles in the Nrf2 knockout and Nrf2 constitutively-activated Keap1 KD (Keap1^{KD/-}) mice livers by microarray analyses as compared with those of the Nrf1-deficient mice livers (FIG. 2A). Among the downregulated genes in the Nrf1 CKO mice, the expression of only a small fraction significantly changed in the Nrf2 knockout or Keap1 KD mice (FIGS. 2B and C). Therefore, the present inventors examined whether the same metabolic pathways that were impaired in the Nrf1 CKO mice were also altered in the Nrf1 knockout or Keap1 KD mice. The results showed that the pathways related to lipid metabolism (FIG. 2D), amino acid metabolism (FIG. 2E), proteasomal degradation (FIG. 2F) and the mitochondrial respiratory chain (FIG. 2G) were not dramatically affected in the Nrf2 knockout or Keap1 KD mice. However, the typical Nrf2 target genes, such as phase II detoxification and antioxidant enzyme genes, were significantly reduced or induced in Nrf2 knockout or Keap1 KD mice, separately (FIG. 2H). Thus, these results strongly suggest that Nrf1 and Nrf2 have distinct roles in hepatic metabolism.

### 1.2.3 Nrf1-Dependent Expression of Metabolic Transcriptional Regulators in Liver

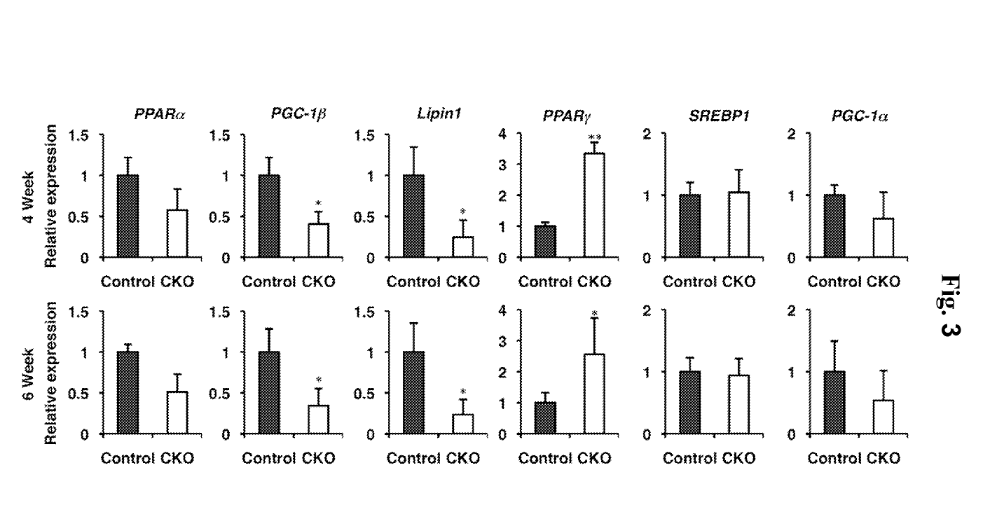

Next, the present inventors characterized the contribution of Nrf1 to the regulation of metabolic pathways. Using microarray analysis, the present inventors observed that the expression of several metabolic transcription factor and coactivator genes, such as PPARα, Lipin1 and PGC-1β, decreased in the livers of Nrf1 CKO mice. To gain insight into the Nrf1-dependent regulation of metabolic pathways, the present inventors examined the mRNA expression levels of these candidates and several regulators involved in hepatic metabolism. Consistent with the microarray analysis data, the expression of PPARα, Lipin1 and PGC-1β decreased in the livers of Nrf1 CKO mice at 6 weeks of age (FIG. 3). In contrast, the expression of PGC-1α and SREBP1 did not significantly change in the control and Nrf1 CKO mice (FIG. 3). The PPARα transcripts increased in the Nrf1 CKO mice as compared with control mice (FIG. 3). Similar results were obtained in the case of 4-week-old Nrf1 CKO mice having no severe hepatic steatosis (FIG. 3). These implicated that Nrf1 is an important regulator of PPARα, Lipin1 and PGC-1β.

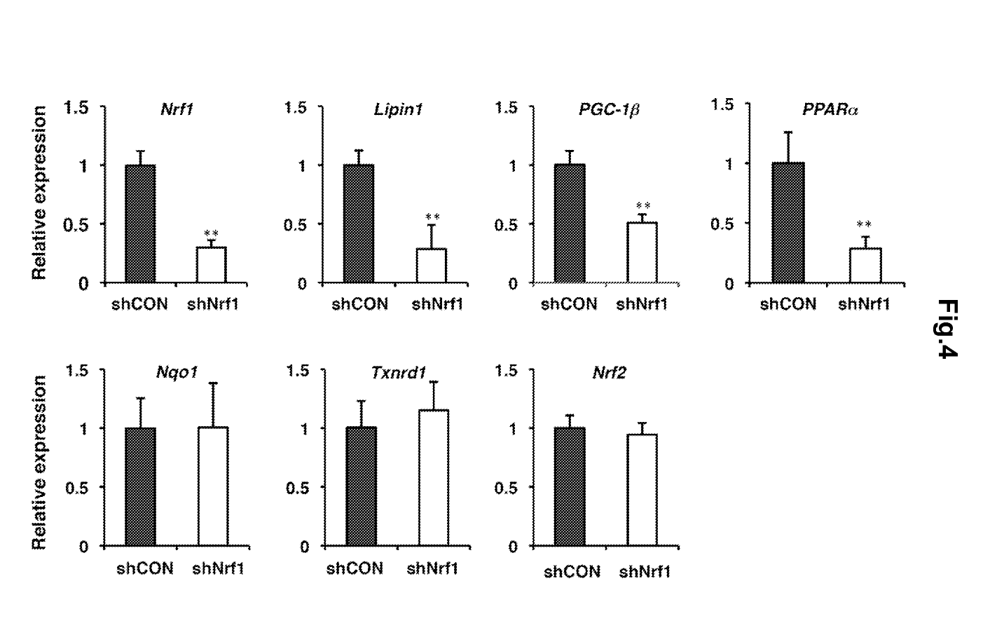

It is also possible that the reduction of PPARα, Lipin1 and PGC-1β mRNA was secondary to hepatic steatosis. To exclude this possibility, the present inventors examined the effect of Nrf1 reduction on the expression of these genes in cell culture system. The present inventors knocked down Nrf1 expression in the mouse hepatoma cell line, Hepa1, using RNA interference-mediated gene silencing. The present inventors prepared five independent cell lines by stably expressing shRNA that specifically targeted to Nrf1, which resulted in the effective reduction of endogenous Nrf1 but not Nrf2 mRNA levels (FIG. 4). Consistent with the data from mouse individuals, the Nrf1 knockdown significantly reduced PPARα, Lipin1 and PGC-1β gene expressions, while the Nrf2 target genes, such as Nqo1 and Txnrd1, did not significantly change (FIG. 4). Collectively, these in vivo and in vitro experiments suggest that PPARα, Lipin1 and PGC-1β are direct or indirect downstream targets of Nrf1.

### 1.2.4 Nrf1 and Small Maf Proteins Bind to Lipin1 and PGC-1β

The previous observation prompted the present inventors to determine whether Nrf1 directly regulates the expression of PPARα, Lipin1 and PGC-1β. The present inventors searched the genomic sequences of PPARα, Lipin1 and PGC-1β and found several putative ARE motifs in their regulatory regions (FIG. 5A). To determine whether Nrf1 and its heterodimeric partner, small Maf, directly bind to these ARE motifs, the present inventors performed ChIP analyses. The chromatin from Hepa1 cells was immunoprecipitated using specific antibodies to each protein, and the amount of precipitated DNA was examined by qPCR. The results showed that Nrf1 was recruited to the AREs in the Lipin1 promoter, and the Lipin1 and PGC-1β introns, but not to those in the PPARα promoter and 5′ UTR region (FIG. 5B). However, MafG, the small Maf protein most abundantly present in liver, was recruited to all AREs tested in this analysis (FIG. 5C). Notably, MafG strongly bound to the AREs in the Lipin1 and PGC-1β introns. This binding might result from the presence of Maf recognition elements (MARE) in AREs that have GC boxes at both ends and are recognized by small Maf homodimers (FIG. 5A). Together, the ChIP analyses strongly suggest that Nrf1 directly regulates the expression of Lipin1 and PGC-1β through the AREs.

### 1.2.5 Nrf1 can Activate Transcription Via the AREs of Lipin1 and PGC-1β

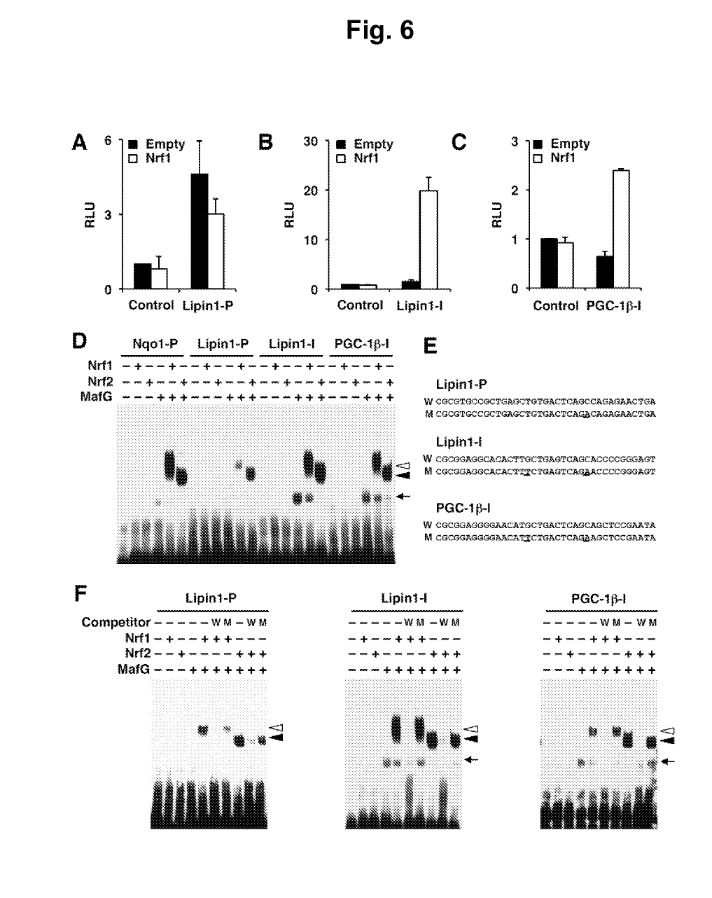

The present inventors further analyzed whether these putative AREs confer Nrf1-dependent transcriptional activity. To this end, DNA fragments containing the ARE motif were inserted upstream of luciferase reporter genes, and the resulting constructs were transfected into Hepa1 cells along with or without the Nrf1 expression vector. The construct containing the ARE of the Lipin1 promoter showed increased basal reporter activity, but no further activation was observed upon addition of the Nrf1 expression vector (FIG. 6A). However, for the reporter constructs containing the ARE of either Lipin1 or PGC-1β introns, the luciferase activities were significantly increased by the expression of Nrf1 (FIGS. 6B and C). These results suggest that Nrf1 can activate transcription through binding to the AREs in the Lipin1 or PGC-1β introns.

To obtain further evidence that the Nrf1-MafG heterodimer binds to the AREs in Lipin1 and PGC-1β, the present inventors performed EMSA. To test the binding specificity of Nrf1 and Nrf2, the present inventors examined the binding of Nrf1-MafG and Nrf2-MafG heterodimers to these AREs and the ARE from the Nqo1 gene. The results showed that both Nrf1-MafG and Nrf2-MafG heterodimers bound to the AREs of the Lipin1 promoter and the Lipin1 and PGC-1β introns (FIG. 6D). The binding affinity of the Nrf1-MafG heterodimer for the ARE of the Lipin1 promoter was slightly weaker than that for the ARE of Nqo1 (FIG. 6D). Particularly, MafG homodimers also bound to the AREs of the Lipin1 and PGC-1β introns (FIG. 6D), and this result was consistent with the ChIP data showing that MafG was strongly enriched in these regions (FIG. 5C). The binding specificity was confirmed by a competition assay using wild-type or mutant oligonucleotides (FIG. 6E). The addition of excess amounts of unlabeled wild-type, but not the mutant oligonucleotides abolished the binding activity of both Nrf1-MafG and Nrf2-MafG heterodimers (FIG. 6F). Thus, these results demonstrate that both Nrf1-MafG and Nrf2-MafG heterodimers bind to the AREs of the Lipin1 promoter and the Lipin1 and PGC-1β introns in vitro.

1.2.6 Nrf2 does not Participate in the Regulation of Lipin1 and PGC-1β.

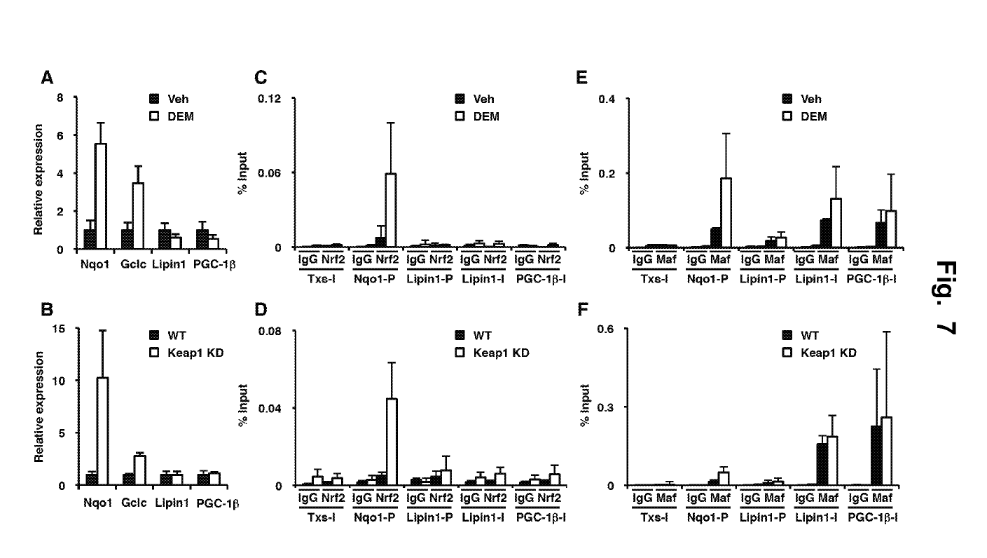

Because the EMSA showed that not only Nrf1 but also Nrf2 could bind to the AREs in Lipin1 and PGC-1β in vitro, the present inventors further examined the possibility that Nrf2 might regulate Lipin1 and PGC-1β. To this end, they treated Hepa1 cells with an Nrf2 inducer, DEM, and examined gene expression by qPCR. While Nrf2 target genes, such as Nqo1 and Gcic, were induced by DEM. Lipin1 and PGC-1β were not significantly affected (FIG. 7A). Similarly, the expression of Nqo1 and Gcic was significantly induced in Keap1 KD livers, but the expression of Lipin1 and PGC-1β did not substantially change (FIG. 7B). Consistent with these expression data, the ChIP analyses clearly demonstrated that Nrf2 was strongly recruited to the ARE of the Nqo1 promoter region, but not to those of the Lipin1 and PGC-1β genes in both the DEM-treated Hepa1 cells and the Keap1 KD livers (FIGS. 7C and D). Again, MafG was recruited not only to the ARE of Nqo1, but also to the AREs in the Lipin1 and PGC-1β gene regulatory regions (FIGS. 7E and F). Collectively, these findings indicate that while Nrf2 could potentially bind to the AREs of Lipin1 and PGC-1β in vitro, endogenous Nrf2 does not participate in the regulation of Lipin1 and PGC-1β in vivo. Taken together, these results show the direct involvement of Nrf1 in the regulation of Lipin1 and PGC-1β, suggesting distinct roles of Nrf1 and Nrf2 in the regulation of hepatic metabolism.

### 1.3 Discussion

Liver-specific Nrf1 knockout mice develop hepatic steatosis. However, it is largely unknown how Nrf1 contributes to hepatic lipid homeostasis. In this Example, the present inventors performed extensive transcriptional profiling of Nrf1-deficient livers and found that an Nrf1 deficiency leads to the dysregulation of several metabolic pathways, such as lipid and amino acid metabolism. The present inventors focused on the transcriptional regulators related to hepaitc metabolism and found that Nrf1, but not Nrf2, directly regulates the expression of the transcriptional coactivators, Lipin1 and PGC-1β. These results suggest that Nrf1 participates in the regulation of hepatic metabolism by controlling the expression of metabolic transcriptional regulators.

Global gene expression profiling revealed that the loss of Nrf1 had a significant effect on various cellular functions. Consistent with findings in previous studies (Literature 2 and 8), the expression of the proteasome subunit genes was downregulated in the livers of Nrf1 CKO mice, suggesting that Nrf1 is actually functional in the liver under homeostatic conditions. This Example also revealed that the pathways related to lipid and amino acid metabolism and the mitochondrial respiratory chain were impaired as a consequence of the Nrf1 deficiency. Notably, the observed dysregulation of the genes involved in amino acid metabolism suggests that Nrf1 contributes to protein catabolism through the regulation of both amino acid metabolism and the proteasomal degradation system. Thus, it is plausible that the overall function of Nrf1 might be to regulate energy homeostasis by inducing the expression of the genes involved in the catabolism of lipids, proteins and amino acids. However, the microarray analyses also revealed that the genes related to cell cycle control and DNA replication were upregulated in the livers of Nrf1 CKO mice. While it is not clear whether these changes are a direct effect of the loss of Nrf1 function, the dysfunction of cell cycle regulation could also contribute to the pathogenesis observed in the livers of Nrf1 CKO mice.

This Example supports the idea that Lipin1 and PGC-1β are direct target genes of Nrf1. Lipin1 was discovered by a positional cloning approach to identify the genetic mutation responsible for the hepatic steatosis dystrophic (fld) mouse phenotype (Peterfy et al., Nat. Genet. 27: 121-124. (2001)). Lipin1 possesses phosphatidic acid phosphatase activity; however, Lipin1 also acts as a coactivator of the PPARα/PGC-1α regulatory pathway and positively regulates oxidative metabolism of fatty acid (Finck et al., Cell Metab. 4: 199-210. (2006)). Fld mice develop neonatal hepatic steatosis associated with the reduced expression of the proteins involved in lipid metabolism and a diminished rate of hepatic fatty acid oxidation (Rehnmark et al., J. Lipid Res. 39: 2209-2217. (1998)). PGC-1β is required for the coactivation of estrogen-related receptor α (ERR α) and nuclear respiratory factor 1 (Vianna et al., Cell Metab. 4: 453-464 (2006)), which are key transcription factors in regulating the respiratory chain reaction through the control of mitochondrial biogenesis. PGC-1β knockout mice show an altered expression of mitochondrial oxidative metabolism genes and high fat diet-induced hepatic steatosis (Vianna et al., Cell Metab. 4: 453-464 (2006); Sonoda et al., Proc. Natl. Acad. Sci. USA. 104: 5223-5228. (2007)). Therefore, the most intriguing possibility suggested by this Example is that the reduced expression of Lipin1 and PGC-1β affects PPARα-, ERRα-, and nuclear respiratory factor 1-mediated transcription, eventually leading to the decreased expression of their target genes and the hepatic steatosis observed in Nrf1 CKO mice. However, it is also possible that the dysregulation of phosphatidic acid metabolism due to the decrease of Lipin1 contributes to the phenotypes in Nrf1 CKO mice.

In addition to the compromised expression of the two metabolic coactivators, it is likely that other abnormalities also contribute to the pathology of the Nrf1-deficient livers. For example, the dysregulation of the methionine metabolic pathway (FIG. 1D) might be related to the progress of hepatic steatosis because a choline-methionine deficient diet induces hepatic steatosis. Interestingly, the genes that were downregulated in the Nrf1 CKO mice included Mat1a and Gnmt, and the loss of these genes typically results in hepatic steatosis (Lu et al., Proc. Natl. Acad. Sci. U.S.A. 98: 5560-5565. (2001); Liu et al., Hepatology 46: 1413-1425. (2007)), although it remains to be determined whether Nrf1 directly regulates these genes. The present inventors assume that the alteration of the gene expression profile in Nrf1-deficient livers reflects the genome-wide compensatory reaction to the loss of Nrf1. Therefore, the present data together with the results from genome-wide Nrf1 binding site studies (i.e., ChIP-sequence analyses) will provide a better overview of the Nrf1-mediated gene regulatory network.

While it is reported that Nrf1 and Nrf2 share functional properties (Leung et al., J. Biol. Chem. 278: 48021-48029. (2003)), this Example suggests that the roles of these two factors are largely different. Consistent with this idea, the present inventors previously reported that Nrf1 selectively regulates the gene expression of metallothionein-1 and -2 in the liver (Literature 7). In addition, this Example strongly suggests that Nrf1, but not Nrf2, directly regulates the expression of Lipin1 and PGC-1β. However, the molecular basis for the differential binding specificity of Nrf1 and Nrf2 is currently unknown. Moreover, EMSA data provided by the present inventors showed that both Nrf1 and Nrf2 could bind to the AREs in Lipin1 and PGC-1β regulatory regions in vitro (FIG. 6D). Nonetheless, the ChIP analyses clearly demonstrated that Nrf1 is specifically recruited to the AREs in Lipin1 and PGC-1β (FIGS. 5 and 7). Therefore, taken together, these observations suggest that additional molecular mechanisms, such as the differential recruitment of transcriptional cofactors or epigenomic differences, including histone modification and DNA methylation, might be involved in the target gene specificity of CNC factors.

In conclusion, the present inventors identified Nrf1 as a novel regulator of Lipin1 and PGC-1β gene expressions. The comprehensive gene expression analyses also showed that the loss of Nrf1 had a significant effect on various metabolic pathways, such as lipid and amino acid metabolism, suggesting that Nrf1 might contribute to energy homeostasis through the catabolism of cellular components. While it still remains unknown how Nrf1 activity is regulated in the liver, it might be possible that nutrition availability influences Nrf1 activity. Thus, the identification of Nrf1 inducers will provide a more comprehensive insight into the function of Nrf1 in the liver.

### Example 2

### 2.1 Materials and Methods

Cloning of mouse Nrf1 cDNA was performed using a method known in the art (Zhang et al., Biochem. J. 399: 373-385. (2006)). An expression vector of full-length mouse Nrf1 and luciferase fusion protein (mNrf1-Luc) was prepared by ligating the sequence, which was amplified by the following primers: 5′-TAA TAC GAC TCA CTA TAG GG-3′ (SEQ ID NO: 21) and 5′-CTT TAT GTT TTT GGC GTC TTC CTT CCT CCG GTC CTT TG-3′ (SEQ ID NO: 22) using mouse Nrf1 cDNA sequence as a template, and the sequence, which was amplified by the following primers: 5′-CAA AGG ACC GGA GGA AGG AAG ACG CCA AAA ACA TAA AG-3′ (SEQ ID NO: 23) and 5′-GAC TCT AGA ATT ACA CGG CG-3′ (SEQ ID NO: 24) using pGL3-basic vector (Promega) as a template; and cloning the resulting sequence at a KpnI/XbaI site of pcDNA3.1/V5His B plasmid (Invitrogen). Furthermore, an expression vector of CNC-bZip domain-deficient mouse Nrf1 and luciferase fusion protein (mNrf1 ACNC-bZip-Luc) was prepared by ligating a sequence, which was amplified by the following primers: 5′-TAA TAC GAC TCA CTA TAG GG-3′ (SEQ ID NO: 25) and 5′-CTT TAT GTT TTT GGC GTC TTC CAT CTG CTT GTC CAG GAA-3′ (SEQ ID NO: 26) using mouse Nrf1 cDNA sequence as a template and a sequence, which was amplified by the following primers: 5′-TTC CTG GAC AAG CAG ATG GAA GAC GCC AAA AAC ATA AAG-3′ (SEQ ID NO: 27) and 5′-GAC TCT AGA ATT ACA CGG CG-3′ (SEQ ID NO: 28) using pGL3-basic vector (Promega) as a template; and cloning the resulting sequence at a KpnI/XbaI site of pcDNA3.1/V5His B plasmid (Invitrogen). The possibility that deletion of CNC-bZip domain would be better to more easily express Nrf1 was conceivable.

Mouse hepatoma cell line Hepa1c1c7 (Hepa1) was cultured in Dulbecco's modified Eagle's medium (DMEM)(Wako) supplemented with 10% fetal bovine serum and 1% penicillin-streptomycin (Gibco). To obtain a cell line expressing mouse Nrf1-luciferase fusion protein or CNC-bZip domain deficient mouse Nrf1-luciferase fusion protein, mNrf1-Luc expression vector or mNrf1 ACNC-bZip-Luc expression vector was linearized by restriction enzyme PvuI, and then transfected into Hepa1 cells using Lipofectamine 2000 transfection reagent (Invitrogen). The cells were cultured in a medium containing 1 mg/mL G-418 and stable expression cells were selected to establish a plurality of clone cell lines.

Cells of each cell line were seeded at a density of 5×10⁴ cells per well in a 24-well plate, cultured overnight and treated with a solvent control, DMSO or 10 μM MG-132 (Peptide Institute) for 4 hours. Thereafter, the firefly luciferase activity was measured using dual luciferase kit (Promega) and a luminometer (Berthold).

### 2.2 Results

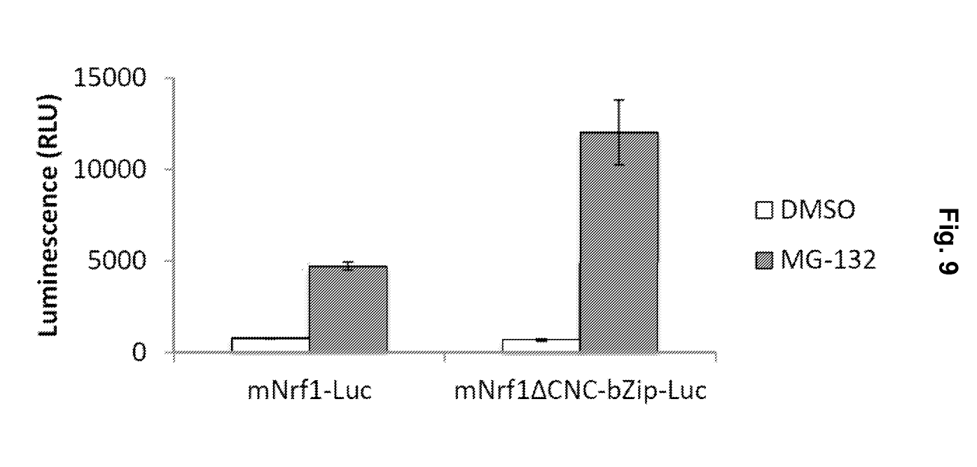

As a result of adding a proteasome inhibitor, MG-132, to stable expression cell lines which mNrf1-Luc expression vector or mNrf1ΔCNC-bZip-Luc expression vector was introduced, a drastic increase of luciferase activity was observed compared to the case where a solvent was added (FIG. 9).

### 2.3 Discussion

Nrf1, similarly to Nrf1, is degraded by a proteasome under static conditions. Upon activation stimuli, Nrf1 protein gets out of the degradation system and stabilized. In this way, Nrf1 is considered to activate transcription of a target gene. Endogenous stimuli and ligand for activating Nrf1 are not elucidated; however, it is reported that Nrf1 protein is stabilized by addition of a proteasome inhibitor and transcription activation ability is induced (Literature 2). This time, an assay system for evaluating stabilization of Nrf1 protein using luciferase activity as an index was constructed by expressing Nrf1 and luciferase fusion protein. As shown in FIG. 9, a drastic increase of luciferase activity was observed by addition of a proteasome inhibitor. From this, it is considered that Nrf1-luciferase fusion protein gets out of its degradation system since the function of proteasome is inhibited, and that the increase in the amount of fusion protein by stabilization is reflected in luciferase activity. From the above, it is considered that the assay system of the present invention using Nrf1-luciferase fusion protein is a functional assay system reflecting endogenous Nrf1 activation mechanism, and that compounds enhancing Nrf1 stability can be screened by using this assay system.

### [Sequence Listing Free Text]

[SEQ ID NO: 1] Nucleotide sequence of cDNA encoding human Nrf1 (Accession No. NM_{—}003204).

[SEQ ID NO: 2] Amino acid sequence of human Nrf1 (Accession No. NP_{—}003195).

[SEQ ID NO: 3] Nucleotide sequence of cDNA encoding mouse Nrf1 (Accession No. NM_{—}008686).

[SEQ ID NO: 4] Amino acid sequence of mouse Nrf1 (Accession No. NP_{—}032712).

[SEQ ID NO: 5] Nucleotide sequence of cDNA encoding human Lipin1 (Accession No. NM_{—}145693).

[SEQ ID NO: 6] Amino acid sequence of human Lipin1 (Accession No. NP_{—}663731).

[SEQ ID NO: 7] Nucleotide sequence of cDNA encoding mouse Lipin1 (Accession No. NM_{—}172950).

[SEQ ID NO: 8] Amino acid sequence of mouse Lipin1 (Accession No. NP_{—}766538).

[SEQ ID NO: 9] Nucleotide sequence of cDNA encoding human PGC-1β (Accession No. NM_{—}133263).

[SEQ ID NO: 10] Amino acid sequence of human PGC-1β (Accession No. NP_{—}573570).

[SEQ ID NO: 11] Nucleotide sequence of cDNA encoding mouse PGC (Accession No. NM_{—}133249).

[SEQ ID NO: 12] Amino acid sequence of mouse PGC-1β (Accession No. NP_{—}573512).

[SEQ ID NO: 13] Nucleotide sequence of ARE region in Lipin1 intron.

[SEQ ID NO: 14] Nucleotide sequence of ARE region in PGC-1β intron.

[SEQ ID NO: 15] Nucleotide sequence including ARE region in Lipin1 promoter (Example 1).

[SEQ ID NO: 16] Nucleotide sequence including ARE region in Lipin1 intron (Example 1).

[SEQ ID NO: 17] Nucleotide sequence including ARE region in PGC-1β intron (Example 1).

[SEQ ID NO: 18] Nucleotide sequence of 19 mer used for preparation of an expression construct of short hairpin RNA (shRNA) targeting Nrf1 (Example 1).

[SEQ ID NO: 19] Nucleotide sequence of a primer used in Example 1.

[SEQ ID NO: 20] Nucleotide sequence of a primer used in Example 1.

[SEQ ID NO: 21] Nucleotide sequence of a primer used in Example 2.

[SEQ ID NO: 22] Nucleotide sequence of a primer used in Example 2.

[SEQ ID NO: 23] Nucleotide sequence of a primer used in Example 2.

[SEQ ID NO: 24] Nucleotide sequence of a primer used in Example 2.

[SEQ ID NO: 25] Nucleotide sequence of a primer used in Example 2.

[SEQ ID NO: 26] Nucleotide sequence of a primer used in Example 2.

[SEQ ID NO: 27] Nucleotide sequence of a primer used in Example 2.

[SEQ ID NO: 28] Nucleotide sequence of a primer used in Example 2.

1. A method for screening a test compound for a compound regulating Nrf1 activity, comprising using Nrf1 and Lipin1 and/or PGC-1β.

2. The method according to claim 1, comprising:
(i) measuring an expression level or activity of Lipin1 and/or PGC-1β in a cell expressing Nrf1 and Lipin1 and/or PGC-1β in a case where the test compound is contacted with the cell; (ii) measuring an expression level or activity of Lipin1 and/or PGC-1β in the cell expressing Nrf1 and Lipin1 and/or PGC-1β in a case where the test compound is not contacted with the cell; and (iii) comparing the expression levels or activities of Lipin1 and/or PGC-1β in the cases (i) and (ii).

3. The method according to claim 2, wherein the test compound is selected as a compound enhancing Nrf1 activity if the expression level or activity of Lipin1 and/or PGC-1β in the case (i) is higher than the expression level or activity of Lipin1 and/or PGC-1β in the case (ii).

4. The method according to claim 1, comprising:
(i) measuring an expression level or activity of a marker gene in a cell, wherein a recombinant vector having the marker gene ligated downstream of an ARE region in a Lipin1 and/or PGC-1β intron is introduced into the cell, in a case where a test compound is contacted with the cell; (ii) measuring an expression level or activity of the marker gene in the cell in a case where the test compound is not contacted with the cell; and (iii) comparing the expression levels or activities of the marker gene in the cases (i) and (ii).

5. The method according to claim 4, wherein the test compound is selected as a compound enhancing Nrf1 activity if the expression level or activity of the marker gene in the case (i) is higher than the expression level or activity of the marker gene in the case (ii).

6. A method for screening a test compound for a compound enhancing Nrf1 stability, comprising using a cell expressing an Nrf1-marker fusion protein.

7. The method according to claim 6, comprising:
(i) measuring an amount of the Nrf1-marker fusion protein in a cell expressing the Nrf1-marker fusion protein in a case where the test compound is contacted with the cell; (ii) measuring an amount of the Nrf1-marker fusion protein in the cell expressing the Nrf1-marker fusion protein in a case where the test compound is not contacted with the cell; and (iii) comparing the amounts of the Nrf1-marker fusion protein in the cases (i) and (ii).

8. The method according to claim 7, wherein the test compound is selected as a compound enhancing Nrf1 stability if the amount of the Nrf1-marker fusion protein in the case (i) is higher than the amount of the Nrf1-marker fusion protein in the case (ii).

9. The method according to claim 1, wherein a compound enhancing Nrf1 stability obtained by screening using a cell expressing Nrf1-marker fusion protein is used as the test compound.

10. A method for screening for a compound promoting lipid degradation, comprising selecting a compound promoting lipid degradation from the compound obtained by the method according to claim 1.


SEQUENCE LISTING (28 sequences)

SEQ ID NO: 1 - DNA, 4891 bp, Homo sapiens
  CDS (652)..(2970)
tttccgagtgcggtagcaggagggggcggggaggtaagcggaggctccgagctctaggccggccggcggtggcggcggcgaggccgggactcgggcttagggcctgctgtggaggcagcggcggacgccgagctaagcagtttctctggaaacccccctggtaagtgtggaggaggcgggacactctgacccaagacgaaaggcctgtagctccagccaaagaaaataaaccttaggagggagaaggaaaaaaaaaatccatcagctgttcctgagaacagcctgcattggaatctacagagaggacaactaatgtgagtgaggaagtgactgtatgtggactgtggagaaagtaagtcacgtgggcccttgaggacctggactgggttaggaacagttgtactttcagaggtgaggtgtcgagaagggaaagtgaatgtggtctggagtgtgtccttggccttggctccacagggtgtgctttcctctggggccgtcagggagctcatcccttgtgttctgccagggtggggtacggggtttgacactgaggagggtaacctgctggctggagcggcagagcagtggccttgatttgtcttttggaagattttaaaaaccaaaaagcataaacattctggtccttcagcaatgctttctctgaagaaatacttaacggaaggacttctccagttcaccattctgctgagtttgattggggtacgggtggacgtggatacttacctgacctcacagcttcccccactccgggagatcatcctggggcccagttctgcctatactcagacccagttccacaacctgaggaataccttggatggctatggtatccaccccaagagcatagacctggacaattacttcactgcccggcggctcctcagtcaggtgagggccctggacaggttccaggtgccaaccactgaggtaaatgcctggctggttcaccgagacccagaggggtctgtctctggcagtcagcccaactcaggcctcgccctcgagagttccagtggcctccaagatgtgacaggcccagacaacggggtgcgagaaagcgaaacggagcagggattcggtgaagatttggaggatttgggggctgtagcccccccagtcagtggagacttaaccaaagaggacatagatctgattgacatcctttggcgacaggatattgatctgggggctgggcgtgaggtttttgactatagtcaccgccagaaggagcaggatgtggagaaggagctgcgagatggaggcgagcaggacacctgggcaggcgagggcgcggaagctctggcacggaacctgctagtggatggagagactggggagagcttccctgcacaggtgcctagtggggaggaccagacggccctgtccctggaagagtgccttaggctgctggaagccacctgcccctttggggagaatgctgagtttccagcagacatttccagcataacagaagcagtgcctagtgagagtgagccccctgctcttcaaaacaacctcttgtctcctcttctgaccgggacagagtcaccatttgatttggaacagcagtggcaagatctcatgtccatcatggaaatgcaggccatggaagtgaacacatcagcaagtgaaatcctgtacagtgcccctcctggagacccactgagcaccaactacagccttgcccccaacactcccatcaatcagaatgtcagcctgcatcaggcgtccctggggggctgcagccaggacttcttactcttcagccccgaggtggaaagcctgcctgtggccagtagctccacgctgctcccgttggcccccagcaattctaccagcctcaactccaccttcggctccaccaacctgacagggctcttctttccaccccagctcaatggcacagccaatgacacagcaggcccagagctgcctgaccctttggggggtctgttagatgaagctatgttggatgagatcagccttatggacctggccattgaagaaggctttaaccctgtgcaggcctcccagctggaggaggaatttgactctgactcaggcctttccttagactcgagccatagcccttcttccctaagcagctctgaaggcagttcttcctcttcttcctcctcctcttcctcttcttcctctgcttcttcctctgcctcttcctccttttctgaggaaggtgcggttggctacagctctgactctgagaccctggatctggaagaggccgagggtgctgtgggctaccagcctgagtattccaagttctgccgcatgagctaccaggatccagctcagctctcatgcctgccctacctggagcacgtgggccacaaccacacatacaacatggcacccagtgccctggactcagccgacctgccaccacccagtgccctcaagaaaggcagcaaggagaagcaggctgacttcctggacaagcagatgagccgggatgagcaccgagcccgagccatgaagatccctttcaccaatgacaaaatcatcaacctgcctgtggaggagttcaatgaactgctgtccaaataccagttgagtgaagcccagctgagcctcatccgagacatccggcgccggggcaagaacaagatggcggcgcagaactgccgcaagcgcaagctggacaccatcctgaatctggagcgtgatgtggaggacctgcagcgtgacaaagcccggctgctgcgggagaaagtggagttcctgcgctccctgcgacagatgaagcagaaggtccagagcctgtaccaggaggtgtttgggcggctgcgagatgagaacggacgaccctactcgcccagtcagtatgcgctccagtacgccggggacggcagtgtcctcctcatcccccgcacgatggccgaccagcaggcccggcggcaggagaggaagccaaaggaccggagaaagtgagcctggggaagaagggggtttgaagcccaccaagaccgaaactggagaagggctggacctggacctggacctggacctacagcggggacttaaatgccttcttatccaatatatcttctcagatgggatgactgcgggtcagtgtacaggaagaggcaggcactggctggctcagctccactcgggtggagtggaagtggccagaccatttagacggacagggtcctcaccctacccctttcctgtgaggcaggggtggtggtggagttgctggaggtagaggagctatgtggagcaaaggccgacagaggggaaggaatggacctgtgagaggaagggaaggtggcagaaagtctcatttcaggaaggagggatagaaggaaggaaggaaggaacccccccccccccgaaaaaaaaatcaaagcgggaagaaaatcagagggaaggttaaggttggctctggccaggattccaggcagcaggttggagtgactggtgggcctagatcactggtgtgataaaccccaattttcaccccggggggggtggggtacacagacacagggtgggggtggggagggacggtgttaactctttctgctccttgcattttgacatccctgaaggggagctcttggatatcattggccatgtttcaatcgaatggagccactgggccccaacactggctttgagatttagagtcaaagggtagagtgaacaggaaagggtcacgtggtcccatgttgcaacagccccaacatcacgcatgtcattcactgccttgccactccatctccctccgtgctccagccacccctgagctgaggctcccattgtctccatcagagcctgcatgtgtatgccgtcctcccctggtccggtgtttgtgttccccacccctcacagactgcctgagctcttctgtaagctggggtagggtgatggcagtgctccgggaactgggcctgcagccttcctcttctgggactgctgtgaggcagaggaatgatggagaatctagtgtagcagcctccaggcaggattcagcacaacactggggagtcacccttccctcgggcctctgcctaccaacaactgggcttatcactgggaaaacacaaaaaattacacaacccagcaacaacaaaagaactagtcctcttagaatttcttgcgctttgatttttttagggcttgtgccctgtttcacttatagggtctagaatgcttgtgttgagtaaaaaggagatgcccaatattcaaagctgctaaatgttctctttgccataaagactccgtgtaactgtgtgaacacttgggatttttctcctctgtcccgaggtcgtcgtctgctttcttttttgggtttctttctagaagattgagaagtgcatatgacaggctgagagcacctccccaaacacacaagctctcagccacaggcagcttctccacagccccagcttcgcacaggctcctggagggctgcctgggggaggcagacatgggagtgccaaggtggccagatggttccaggactacaatgtctttatttttaactgtttgccactgctgccctcacccctgcccggctctggagtaccgtctgccccagacaagtgggagtgaaatgggggtggggggaagcactgattcccagttagggggtgcctaactgagcagtagggatagaaggtgtgaacctgggagtgcttttataaattattttccttgtagattttatttttaatttatctctgtgacctgccagggagaggggagagagagagagatgctgttgagcacatgacaaaataaaataaaatggatgattcattcttaagtgcactttttccccactttgaatttaaattgagaataaaggaaatggactcattgtaaaaaaaaaaaaaaaaaa

SEQ ID NO: 2 - protein, 772 aa, Homo sapiens
MLSLKKYLTEGLLQFTILLSLIGVRVDVDTYLTSQLPPLREIILGPSSAYTQTQFHNLRNTLDGYGIHPKSIDLDNYFTARRLLSQVRALDRFQVPTTEVNAWLVHRDPEGSVSGSQPNSGLALESSSGLQDVTGPDNGVRESETEQGFGEDLEDLGAVAPPVSGDLTKEDIDLIDILWRQDIDLGAGREVFDYSHRQKEQDVEKELRDGGEQDTWAGEGAEALARNLLVDGETGESFPAQVPSGEDQTALSLEECLRLLEATCPFGENAEFPADISSITEAVPSESEPPALQNNLLSPLLTGTESPFDLEQQWQDLMSIMEMQAMEVNTSASEILYSAPPGDPLSTNYSLAPNTPINQNVSLHQASLGGCSQDFLLFSPEVESLPVASSSTLLPLAPSNSTSLNSTFGSTNLTGLFFPPQLNGTANDTAGPELPDPLGGLLDEAMLDEISLMDLAIEEGFNPVQASQLEEEFDSDSGLSLDSSHSPSSLSSSEGSSSSSSSSSSSSSSASSSASSSFSEEGAVGYSSDSETLDLEEAEGAVGYQPEYSKFCRMSYQDPAQLSCLPYLEHVGHNHTYNMAPSALDSADLPPPSALKKGSKEKQADFLDKQMSRDEHRARAMKIPFTNDKIINLPVEEFNELLSKYQLSEAQLSLIRDIRRRGKNKMAAQNCRKRKLDTILNLERDVEDLQRDKARLLREKVEFLRSLRQMKQKVQSLYQEVFGRLRDENGRPYSPSQYALQYAGDGSVLLIPRTMADQQARRQERKPKDRRK

SEQ ID NO: 3 - DNA, 4654 bp, Mus musculus
  CDS (667)..(2892)
gagtgcagtggaaggagggggcggggagggaagccgaggctgggcgctccaggccggccggaggtggcggcgacggcggcggtggcgaggccgggacttgggctgagggcctgctgtggcggcagcgacagactgtgacctcagcagcggggttctctggaaacccccctggtaagtgcagaggaggcgggacactctgacccaagacaaagacctgtagctccagccaaagaaaataaaccttaggaaggagaagggggggaaaaaaaaaaggaggaaagaaagactccatcagctgttggtgtgagcagcctgcagtggatctccatagaggacaactaatgtgaatccgaaagtgactgtgtatgggctgtggtgaagtaagttgagggccctgagcgcctggactgtgttaggaacgatcgaacggctcaactttgcgaggtgaggtgtcaaaaagggaaaagtgaatgtggctttcgctccacggggtgtgctgtcgtctggggccgtcagggagctcagcccttgtgttgtgccagggtggggtccagggtctggcactgaggagggtagcctgctggctgaagtggcagagcagtggccttgatttgtcttgtggaagatttaaaaacaaaaaagcataaatattctggtccttcagcaatgctttctctgaagaaatatttaacggaaggacttctccagttcaccatcctgctgagtctgattggggttcgggtggacgtggatacttacctgacctcacagctcccccctctccgggagatcatcctggggcccagctctgcctatacccagacccagttccacaacctgaggaataccttggatggctatgggatccaccccaagagcatagacctggacaattacttcactgcccggcggctccttagtcaggtgagggccctggataggttccaggtgcctaccactgaggtcaatgcttggctggtccaccgagacccggaggggtctgtctctggcagccagcccaactcaggcctcgccctcgagagttccagtggcctccaagatgtgacaggcccagacaacggggtgagagaaagcgaaacggagcagggattcggtgaagatttggaggacctgggggctgtagcccctcctgtcagtggagacttaaccaaagaggatatagatctgattgacatcctttggcgacaggatattgatctgggggctgggcgtgaggtttttgactacagtcatcgccagaaggagcaggatgtggataaggaactgcaagatggacgagaacgagaggacacctggtcaggcgagggtgcggaagctctggcccgagacctgctagtagatggagagactggggagagcttccctgcacagttcccagctgacgtttccagcatcccagaagcagtgcctagtgagagtgagtcccccgcccttcagaacagccttctatctcctcttctgacggggacagaatcaccatttgatttggaacagcagtggcaagatctcatgtccatcatggaaatgcaggctatggaagtaaatacatcagcaagtgagattctgtacaatgcccctcctggagaccctcttagcaccaactacagccttgcacccaacactcccatcaatcagaatgtcagcctgcatcaggcgtccctggggggctgcagtcaggacttctccctcttcagccccgaggtggagagcctgcctgtggctagcagctccacactgcttccactcgtccccagcaactccaccagtctcaactccacctttggctctaccaacctagcagggcttttctttccatcccagctcaatggcacagccaatgacacatcaggccctgagctacctgacccccttgggggcctgttagacgaagctatgctggatgagatcagcctgatggacctggccattgaggagggcttcaacccggtgcaggcttcccagctcgaagaggagtttgactctgactcaggcctctccttggactccagccatagcccttcctctctgagcagctctgaagggagctcttcttcttcctcctcctcctcttcctcttctgcttcctcctctgcctcttcttccttctctgaggagggtgctgttggttacagctctgactctgagaccctagacctagaagaggctgagggtgcagtgggctaccagccggaatactccaagttctgccgcatgagctatcaggatccttctcagctctcttgccttccctacttagagcatgtgggccacaatcatacatacaatatggcacccagtgcccttgactctgctgatctaccaccacccagcaccctcaagaaaggtagcaaggaaaagcaggctgacttcctggacaagcagatgagccgagatgagcacagagcccgagccatgaagatcccattcaccaatgacaagatcatcaacctgcctgtagaagaattcaatgagctgctgtccaaataccagctgagcgaggcccagctcagcctcatccgggatatccggcgccggggcaaaaacaagatggctgcacagaactgccgcaagcgcaagttggacaccatcctaaacctagaacgtgatgtggaggacttgcagcgagataaggcccgattgcttcgagaaaaggtagagttccttcggtctctgcgacagatgaagcagaaggtccaaagcttataccaggaggtgtttgggcggctgcgggatgagcatgggaggccctactcacccagtcagtatgcccttcagtatgctggggatggcagtgtcctcctcattcctcgcacgatggctgaccagcaggctcggcgacaggagagaaagccaaaggaccggaggaagtgagcctggggaggcagggggtggacgctcactaagaccgaaactggagaagggctgggcctggacctaacattggggacttaaatgccttcttatccaatatatcttctcagatgggatgactgcgggtcagtgcaccgaagaggcgggcgcaggcgctgtctggctcagctgcccccttggggtgggcagggaggaccagactgcttgggtgattggggtccccagcctattccctttctcttgaggggagggtagtgtcggcatgctggaagtagaggagctgtgtggagtgaaggagagaaagtgtgggagatctcattgctggaaggagaaaaggaaggaatcccccgaaaatcaaagcagtcagaaaaaccagagcgactgttaagggctttggccagctttctaggcagcgagtgcaggtgacaacggtggtctagggagagttactggtatggaacacagacatggcggggccccagaaggcctttgtaactgtttcttcaactcttgcatccctgaagggaagatgctcttggatgcacctgtaatatcttagttactgaatgggaagctgtaggggccgaggagggcagagggtataggaagtgagaacgaggcctgtgtcgcagcagcccagcatcaagcatgtcacacactgccctgccacagccacctccctccctggccatcccagagccgaggctcccactgtcctcagagagcctgcatggaaatgctgtcctcttccactctcctcctctttttgatacccaccctcactagctgcctccagctctggagtggggtgctattctggcagtatctggaacttggcctacagcttcctctgcagggtctaaacagggaaggcacgtgtggaggagtggtcccagtgacatccaggcaccattcagcacaacactgggaagtgattcttccctcaggcccctctgcctaccaacacctgggctcctcactgggggaaacaaaagcctataaaccccagcaacaaaacctagtcctcttagacgttcttgcgctttgatttttttagggcgtgtgccctgttacacttatagggcctaggatgcttgtgttgagtaaaaaggagatgccccaatattcaaagctgctaaatgttctctttgccataaagactccgtgttaactgtgtaaacacttgggatttttctcctatgtcccgaggtctggtcttgatttcttttttgggtttctttctaggaaaatgagaagtgcatgcaaggggcaggagatgaccctcccctaggctttcagcttcaggcagcttcttcacagcctgttcagcctgggctcctggaggacagccctgggggaggcagtgaggggcagcgccaagatagccaggtggttggttccaggaccacagtgtcttttttttgttgttggttttttgttgttgttgtttgtttgtttgttttttaactgccactgccgccccctgacccccaatcttggtcagctctggagtactgcctgccccagacgagcaggggttgggggggagcactgatcctcctccctgggcagggcagagggctttcctaaccgagcagtagggatagaaagcgtgagcctgggagtgctttttataaattattttccttgtagattttatttttaatttatctctgtgacctgccagggagaggagagaaagaaatgctgtgagcacatgacaaaataaaatcaaataaaatggatgattcagcttaagtgc

SEQ ID NO: 4 - protein, 741 aa, Mus musculus
MLSLKKYLTEGLLQFTILLSLIGVRVDVDTYLTSQLPPLREIILGPSSAYTQTQFHNLRNTLDGYGIHPKSIDLDNYFTARRLLSQVRALDRFQVPTTEVNAWLVHRDPEGSVSGSQPNSGLALESSSGLQDVTGPDNGVRESETEQGFGEDLEDLGAVAPPVSGDLTKEDIDLIDILWRQDIDLGAGREVFDYSHRQKEQDVDKELQDGREREDTWSGEGAEALARDLLVDGETGESFPAQFPADVSSIPEAVPSESESPALQNSLLSPLLTGTESPFDLEQQWQDLMSIMEMQAMEVNTSASEILYNAPPGDPLSTNYSLAPNTPINQNVSLHQASLGGCSQDFSLFSPEVESLPVASSSTLLPLVPSNSTSLNSTFGSTNLAGLFFPSQLNGTANDTSGPELPDPLGGLLDEAMLDEISLMDLAIEEGFNPVQASQLEEEFDSDSGLSLDSSHSPSSLSSSEGSSSSSSSSSSSSASSSASSSFSEEGAVGYSSDSETLDLEEAEGAVGYQPEYSKFCRMSYQDPSQLSCLPYLEHVGHNHTYNMAPSALDSADLPPPSTLKKGSKEKQADFLDKQMSRDEHRARAMKIPFTNDKIINLPVEEFNELLSKYQLSEAQLSLIRDIRRRGKNKMAAQNCRKRKLDTILNLERDVEDLQRDKARLLREKVEFLRSLRQMKQKVQSLYQEVFGRLRDEHGRPYSPSQYALQYAGDGSVLLIPRTMADQQARRQERKPKDRRK

SEQ ID NO: 5 - DNA, 5363 bp, Homo sapiens
  CDS (68)..(2740)
cagccggcggcgggctgggtgtttgcaatacaaaggcggccacgcgcggcgccgctcggtgcagaccatgaattacgtggggcagttagccggccaggtgtttgtcaccgtgaaggagctctacaaggggctgaatcccgccacactctcagggtgcattgacatcattgtcatccgccagcccaatggaaacctccaatgctcccctttccacgtccgctttgggaagatgggggtcctgcgctcccgagagaaagtggttgacatagaaatcaatggggaatctgtggatttgcatatgaaattgggagataatggagaagcattttttgttcaagaaacagataatgatcaggaagttatccctatgcacctggccacctcccccatcctgtcagaaggagcttcgagaatggaatgccagctgaaaaggggctctgtggacaggatgagaggcctggaccccagcacgccagcccaagtgatcgctcccagcgagacgccgtcaagcagctctgtagtaaagaagagaagaaaaaggaggagaaagtcacagctggacagcctgaagagagatgacaacatgaacacatctgaggatgaggacatgttccccatcgagatgagctcggatgaggccatggagctgctggagagcagcagaactcttcctaatgatatacctccattccaagatgatattcctgaggaaaacctctccctggctgtgatttaccctcagtcagcctcataccctaattcggatagagagtggtcacccactcccagtccttccggttcccgaccttcaacacctaaaagtgattcagaattggtcagcaagtccacggaaaggacagggcagaagaacccagaaatgctttggctgtggggagagctgccgcaggctgctaagtcttcttctccacacaagatgaaagagtccagcccattgagcagtagaaaaatttgtgataaaagtcactttcaggccattcacagcgaatcttcagacacttttagtgaccaatcgccaactctggtcggtggggcacttttggaccagaacaagcctcagacagaaatgcagtttgtgaatgaagaagacctggagaccttaggagcagcagcgccactcttgcccatgatcgaggagctcaaacccccctctgccagtgtagtccagacagcaaacaagacggattctccttccaggaaaagagataaacgaagccgacatcttggtgctgacggcgtctacttggatgacctcacagacatggatcctgaagtggcggccctgtattttcccaaaaacggagatccttccggactcgcaaaacatgcaagcgacaacggagcccggtcagccaaccagtccccgcagtcggtgggcagctcgggcgtggacagtggcgtggagagcacctcggacgggctgagggacctcccttccatcgccatctccctctgcgggggcctcagcgaccaccgggagatcacgaaagatgcattcctggagcaagctgtgtcatatcaacagtttgtggacaaccccgctattatcgatgaccccaatctcgtggtaaagattgggagtaaatattataactggacaacagcagcacccctcctcctggcaatgcaggccttccagaaacctttgccaaaggccactgtggaatctatcatgagggataaaatgcccaaaaagggaggaagatggtggttttcatggaggggaagaaacaccacaatcaaggaggaaagtaagccagagcagtgcttggctggcaaggcccatagcaccggagagcaaccgccgcagctcagcttggccaccagggtaaagcatgaatcatcctccagtgatgaggagcgcgcagctgccaagccatcaaacgcaggccacctccctcttctgcctaatgtcagctacaagaagactctccggctgacttccgagcagcttaaaagcttgaagttgaagaatggccccaacgacgtggttttcagtgtcaccacgcagtaccaaggcacgtgccgctgtgagggcaccatctatctgtggaactgggatgataaagtcatcatttctgatattgatgggacaattaccagatcagatactcttggccacattttgcccacccttgggaaggattggacccatcagggcatcgctaagctgtaccataaagtgagccagaatggatataaatttctctactgttctgcccgtgccatcgggatggcggacatgacgcggggctacctgcactgggtcaacgagaggggcacggtgctgccccaggggcccctgctgctgagtcccagcagcctcttctctgccctgcacagagaagtgattgaaaagaagccagaaaagtttaaagtccagtgtttgacagacatcaaaaacctgtttttccccaacacagaacccttttatgctgcttttggaaaccgaccagctgatgtgtattcatacaagcaagtaggagtgtctttgaatagaatatttaccgtcaaccctaaaggagagctggtacaggaacatgcaaagaccaacatctcttcgtatgtgagactctgtgaagtagtcgaccacgttttcccgttgctgaaaagaagccattcttcagactttccctgttcggataccttcagtaacttcaccttttggagagagccactgccaccttttgaaaaccaggacattcattctgcctcagcgtaaaatgtcccaagcagcctcttgccagcagtgcagagcctggttgtcacccattaaaggataggtctccccggagtgcacagctccacctgggagcctggcgcgtcatcattggcctgacagcagagagaattgagaagcatttctcccctgccccaccccggggctgacatttctaagcaagataggaagggagcactttctaggctaggagttgggtgcatttgtaccgtgaaaagcattcctcagttgtggcttaatgccagttacgacgctgcctttccggcctgctccagcaagtagctactggttcacgtgcagtttggggctgtgaaacctaggcagaaggcggctgtctgagggctgtccccgcctaggacagggtcaatcgaggaatgccagatgtgcacggtttttggcaaagtagggggcacatttccattatagcaatgttagtgccaccaccttctgaacacagtggggagggctgtgaaggctcatgtgacctggatctgaggtctctgatagaaatctggacgccaccgggtccaggcctggcctcagacttggccttgtggatgggccccttacagtatttgctgactagtctcatttttaggtgataaatttttctttaattcctttggttaaagatagtctatttcattggcatatctccccccagtttttgtggctcaaggctggaatatttatgccttaatatatctatggcagacatttaagaatgcgctttatctagctcatggtaactttgcaacgccttagattaaaatgacagtaaatattactaaggcagtattttgaatgagtttgacactgccggcttccttccatccagcgaggtggtgctgacagtgtggacttgagcacacttatgccaaatgataatgatactgacttctgttgggagctctccaaagaaactggttggttttaagaaaatagtttcaagaagttcaactatattcttttagatattatgtattgttttactctgattaggttactgtgataggcatttattcatattctttctataccactgtcattaatatattaaaaagatgtatgtgttagactatcgaaagggccttattctctctttctcatagactgaccttcttttggaatttctgagtcatttattttccttagctttttccactcaaattaagggcaagcgaaaaagtaataatttggcattctttaagcctacagaatgtgattctttcacttgtttattacactggctcgtggacagaacaatttgaaaagtgaaagaattattttggtaaaagattttgctttacttttcgaagcattatttttttaaagagtgttttactccaacgattgaaacattttcctatttaaatttcattgttagaatcacaggaggcaaaaaatggaacggttgaatgaaattttactctttctgtgaaagaaaatccacagagttgttgcctccgttgtagttggtgggccccgttagcattggatgcctttgccaaatggttcatgtggacacacaaaggcaaacagatctgccatcgatcgcagatttctgtagaaacacggatgtgcatgtgcagattcccttttgcaggtattaaaaataattaaaaatagtcctgcctgaggttgcagtgagccgagcttgcactactgcactccagcctgggtgacagagtaagactccatgtcaaaaaaaaaaaaaaaaaaaaaaaagtcctgccttaactaactcctctgcgcttgttcactagtaacctaaagaggctatattcattctttatgcaatgagggtatttttgagtgaattttaactgctctgaactaagtataagctcatgggcctgcaaaggttcagacggtttctcctttgcacccaggaggaactttggctgcgagaatggggggatgtatccctcatgcagttggcatccaggcagccctctgcagcagcacaccctgcaggcggagttttcagaggatgcaattttggatcccgaattttgatgtaccttaaacttccacatcactgcaccctgaaacagagcatgctttccagaaagtcacactctcagatctgtgtcaagttcaatgtgagccctggcaaggctggcatattaacacctgccttctggcttctgaaagtgagatttgtatatgggctgcactcacgcatatacgagttggtttatctttgtgtacatgactataacccagtgatgctgaggtcatgtgctggaatgctgtatttggaccacacatttcaaagttgccctatggaaatgaatcctacttagtgacaagtcatcaaatgtttgtcacatgtgatgaagacaaatatgtatacctggcatagagaaaaatatatacctggtacattggagaaaaataattacactttcaaagagaattccctttgcaattttatgtttggatcaccactgtaagcacactttatttgcatttgatctgtatttgtatatgctgatgcaatgataaaaatcactgtaatacttcattgtgttgtactggatgcaaagctagaaaatattgcaataaatgagaccgatgaaagacttctctgaaaaaaaaa

SEQ ID NO: 6 - protein, 890 aa, Homo sapiens
MNYVGQLAGQVFVTVKELYKGLNPATLSGCIDIIVIRQPNGNLQCSPFHVRFGKMGVLRSREKVVDIEINGESVDLHMKLGDNGEAFFVQETDNDQEVIPMHLATSPILSEGASRMECQLKRGSVDRMRGLDPSTPAQVIAPSETPSSSSVVKKRRKRRRKSQLDSLKRDDNMNTSEDEDMFPIEMSSDEAMELLESSRTLPNDIPPFQDDIPEENLSLAVIYPQSASYPNSDREWSPTPSPSGSRPSTPKSDSELVSKSTERTGQKNPEMLWLWGELPQAAKSSSPHKMKESSPLSSRKICDKSHFQAIHSESSDTFSDQSPTLVGGALLDQNKPQTEMQFVNEEDLETLGAAAPLLPMIEELKPPSASVVQTANKTDSPSRKRDKRSRHLGADGVYLDDLTDMDPEVAALYFPKNGDPSGLAKHASDNGARSANQSPQSVGSSGVDSGVESTSDGLRDLPSIAISLCGGLSDHREITKDAFLEQAVSYQQFVDNPAIIDDPNLVVKIGSKYYNWTTAAPLLLAMQAFQKPLPKATVESIMRDKMPKKGGRWWFSWRGRNTTIKEESKPEQCLAGKAHSTGEQPPQLSLATRVKHESSSSDEERAAAKPSNAGHLPLLPNVSYKKTLRLTSEQLKSLKLKNGPNDVVFSVTTQYQGTCRCEGTIYLWNWDDKVIISDIDGTITRSDTLGHILPTLGKDWTHQGIAKLYHKVSQNGYKFLYCSARAIGMADMTRGYLHWVNERGTVLPQGPLLLSPSSLFSALHREVIEKKPEKFKVQCLTDIKNLFFPNTEPFYAAFGNRPADVYSYKQVGVSLNRIFTVNPKGELVQEHAKTNISSYVRLCEVVDHVFPLLKRSHSSDFPCSDTFSNFTFWREPLPPFENQDIHSASA

SEQ ID NO: 7 - DNA, 5482 bp, Mus musculus
  CDS (63)..(2738)
gtttgcatacaaaggcagccacacgcgtgcgcgcctgctcgtgaatcctcttggttcagacaatgaattacgtggggcagctggccggccaggtgtttgtgacggtgaaggagctctacaagggactgaaccccgccacgctgtcgggatgcatcgacatcattgtcatccggcagcccaatggcagtctgcagtgctcccctttccacgtccgcttcggcaagatgggtgtcctccgctcccgagagaaagtggtggacatagaaatcaatggggagtccgtggatttgcacatgaagttgggagacaacggagaagcattttttgttcaagagactgacaacgatcaggaaatcatccccatgtacctggccacgtcccccatcctgtcagaaggagctgcgagaatggaaagccagctgaagaggaactctgtagacagaatcaggtgcctggatcccactacagctgcccagggcctgcctcccagcgacaccccatccactggttctctggggaagaagagaaggaaaaggaggaggaaggcccagttggacaatctcaaaagagatgacaatgtcaacacatctgaggatgaggacatgtttcccatagagatgagctcggatgaggacacagcaccgatggatggaagcagaactcttcctaatgatgtaccaccattccaagatgatattcctaaggaaaacttcccctcgatttcaacgtacccccagtcagcatcgtatcccagttcggacagagaatggtcccccagccccagtccttcaggctcccggccctcaacaccaaaaagtgactctgagctggtcagtaagtctgcagacaggttgacgccaaagaataacctggaaatgctctggctgtggggtgaattgccacaggctgcaaagtcctcttctccacacaagatgaaagagtccagccccttagggagccggaagactcctgataaaatgaattttcaggccattcacagcgagtcttcagatacttttagtgaccagtcgccaacaatggcccggggactgctcatccaccagagtaaggcccagacggaaatgcagtttgtgaacgaggaggatctcgagtccttgggggcggcagccccaccttcacccgtggccgaagagctcaaggccccatatcccaacaccgcacagtcgtcgagcaagacagattccccttccaggaagaaagataaacggagccgacaccttggagctgatggtgtttatctggacgacctcacggacatggaccctgaagtggcagccctgtatttccccaagaatggggatcctggtgggctccccaaacaagccagtgacaacggagccaggtcagccaaccagtcaccacagtctgtgggaggctcgggcatcgacagtggtgtggagagcacctccgacagcctgagggacctgccatccatcgccatctccctctgcggtggcctcagtgaccacagagagatcaccaaagatgcatttttggaacaagccgtgtcatatcagcaatttgccgacaaccctgctatcatcgatgaccccaacctcgtggtcaaggttggcaataagtattacaactggacaacagcagctcctctacttctggcgatgcaggctttccagaaacctttgccaaaggccactgtggaatccatcatgagagataagatgcccaaaaagggaggaagatggtggttttcctggagaggaagaaatgccacaatcaaagaggaaagcaagcctgaacagtgcctgactgggaaaggccacaataccggagagcagcctgcccagcttggcctggccaccaggataaagcatgagtcatcctccagtgatgaagagcacgcagccgccaagccatcaggttcgagccacctctctctcttgtccaacgtcagctacaaaaagaccctgcggctcacgtcggagcagctgaaaagcttgaagttgaagaacggccccaatgatgtggtgttcagtgtcactacccagtaccagggcacctgtcgctgcgagggcaccatctacctgtggaattgggacgacaaagtcatcatctcagatatcgatgggaccatcacaagatctgatactcttggtcacattttgcccacgctgggaaaggattggactcaccagggcattgcaaagctgtaccacaaagtaagccagaatggctacaagtttctctattgttcggcacgtgccattgggatggcggacatgacgaggggctacctgcactgggtcaacgagaggggcacggtgcttccacagggcccgcttctgctcagcccgagcagcctcttctccgccttgcacagagaagtgattgaaaagaagccagaaaagttcaaagtccagtgtttgacagacatcaagaacctgtttttcccaaacacagaacccttctatgctgcttttgggaaccggcctgctgatgtgtattcctacaagcaagtgggagtgtccctgaataggatcttcactgtgaaccccaagggtgagctggtgcaggagcatgccaagaccaacatcagctcgtacgtgcggctctgcgaagtggtcgatcacgtcttcccattgctaaagagaagccattcctgtgacttcccctgttcagacactttcagtaacttcaccttttggagagagccactgccaccttttgaaaaccaggacatgcattcagcctcagcttgacgcgaccgagcattaaaggataggttgtggggaccctggagctgctgggaaggctgatatgtggccacctgcctaagagagaagcatttctccctcggctcgctcgctcgctcgctcgctcgccccagggtgacacttctaagcatggagggcggagagaggctgcatctcatcccataggctgcaagaggattgggtgcactaggagtgtgcagagcagccctctgtttgggtttagcacacctcatgctgcaccctccagtcccgtctgctccagcaattagttaatgtgcaatacgggtgatgaccccttatcagcgggcaggccacctgagagcctgtctcagtatcactgtgcccctaccccctggaactctgatgtgcatagggatcagtagcatcagggacacatttccacgacagagacatccatgccaccttctgcacacaggctgtgtcccgagtctaagtgaagggaacatgggcctcgctgggtctcagccctagccttggctttgactttttagacgttcccttgcagtattgccgactgtctcactttaggtgataagctctttaattgctttggttaaaggtggcctgtgacgcagctgcatctctccccattcctcatagctcaaggctgggctatttatgccttaacgcatctgcagcaggcatttccttagcgtgcactgtctagctctccgtaactgcaatgccttaccttacaagggtagtccctaaggcagcgtgtgtccgcggcatctcagcaccttctgcgtcctgctacccagcaaggaggagccactagtgtggactcagcagcacttacgcccaccaggaaggatgccgctaaagacactggttgtttttttttaagaaacatagtttccggaagttaaattattttctttgcagatattatttatttctttactgtcactgggctgctgttgtgagcatcctagccagatccttaggcttatagccgtcatgaatgcattaaaagttaagtgtgatagacagtccaaagagcctcattctgtggatgtcacactctgatcttctagaatctccgaatcactcatttttcttagttttttttccccccacagtctaagggaaagaaaaaaaagtaataatttgagcaatgttctttaagctgatagaaggttgctgtgaactgtctcagaacaatgtgaaaaatgaaaagtattttgctaaaagagttgctttactttgaaagtattatttttcttaaagggtgttttactccaaagacagatctttctatttaaatttcattgttagggtcagaggagacaggaacaaaaaaaaaaaaaaaaaaaggaaaagatagaaagaagagttaaaaaagaaaaaaagcagtagaatggactgttactctttctgtgaaagaaaacctagagtcactgcccttgtagtcactgggctctgttaaggtctcatgtggtccacatagttcacaggaatgtaatgaaagccactgtcatctgctgtgggcgcccactgatggagaaactcggccctgagtgtgtggctccctctgggagtcaggaaagccattaagctcctggcttgttctctcgtgacctttggaggccatggtaacccttctgccaccacggtggttttgtgtgtattttgattgctgtggactgtatacgagctcactggcctgcagaggttcacggccgtctccctcatgcccaggggaagctctggcatctcttttcaaatgccctctgcagcccgctgtccaggtggctccctgatgccatgttaggacttgagccagagtctctatagccaaggctggtctacaatgcaccatcttagacatacagcagtccttccacctggcttgggaacctagttttgatataccttaaacttccctgtcactctaccctgaaacagagcacacctcccacaaaaccacactctatgatctctgtgaaaatcttcaggagccctgtccaggcagtgatgtggacgtctactgttaggctccctgtgagatccaaatgagagctgcaccccggttctctcaagttgcttatttgcatctgcacttaaacccagaggccccaaggtcacgtgttaggatactatatgggtaccatacatttcaaagttgctcatggaaatgagcgacttgctccatagtaacatgtcctcaaatggcgatcccacatgatgaaggagaacccacctggcgcatctgagacaggcttctgtcgtcagatactttccttttggatttcaggttcggatcactacttcagaatattttacttacaacgttggctgtgtgtttgcaaatgccggtgcgatgattaaaaaaaaaatcaatataatatttcactgtgttgtgcttgatgcagagctagaaatttctgtaataaacaagatggatgaaagacttctgaaaatcaggcatatgaactttaaattatttttataagttcttaaggtgctcttatttttttctggtttcttatatacaatacttagttatttacttgaagggggaaaaaacccttagtgtgggctagggaaatggctcaggcagtaagagtacttgccatgcaagcaagcacatccaagttctatccccattatccatgaatgaggcctaatgacagtgtgcattgttataccagtgcccgggagatgaggatgggtggatccgtggtgctggctagacagacagccca

SEQ ID NO: 8 - protein, 891 aa, Mus musculus
MNYVGQLAGQVFVTVKELYKGLNPATLSGCIDIIVIRQPNGSLQCSPFHVRFGKMGVLRSREKVVDIEINGESVDLHMKLGDNGEAFFVQETDNDQEIIPMYLATSPILSEGAARMESQLKRNSVDRIRCLDPTTAAQGLPPSDTPSTGSLGKKRRKRRRKAQLDNLKRDDNVNTSEDEDMFPIEMSSDEDTAPMDGSRTLPNDVPPFQDDIPKENFPSISTYPQSASYPSSDREWSPSPSPSGSRPSTPKSDSELVSKSADRLTPKNNLEMLWLWGELPQAAKSSSPHKMKESSPLGSRKTPDKMNFQAIHSESSDTFSDQSPTMARGLLIHQSKAQTEMQFVNEEDLESLGAAAPPSPVAEELKAPYPNTAQSSSKTDSPSRKKDKRSRHLGADGVYLDDLTDMDPEVAALYFPKNGDPGGLPKQASDNGARSANQSPQSVGGSGIDSGVESTSDSLRDLPSIAISLCGGLSDHREITKDAFLEQAVSYQQFADNPAIIDDPNLVVKVGNKYYNWTTAAPLLLAMQAFQKPLPKATVESIMRDKMPKKGGRWWFSWRGRNATIKEESKPEQCLTGKGHNTGEQPAQLGLATRIKHESSSSDEEHAAAKPSGSSHLSLLSNVSYKKTLRLTSEQLKSLKLKNGPNDVVFSVTTQYQGTCRCEGTIYLWNWDDKVIISDIDGTITRSDTLGHILPTLGKDWTHQGIAKLYHKVSQNGYKFLYCSARAIGMADMTRGYLHWVNERGTVLPQGPLLLSPSSLFSALHREVIEKKPEKFKVQCLTDIKNLFFPNTEPFYAAFGNRPADVYSYKQVGVSLNRIFTVNPKGELVQEHAKTNISSYVRLCEVVDHVFPLLKRSHSCDFPCSDTFSNFTFWREPLPPFENQDMHSASA

SEQ ID NO: 9 - DNA, 10642 bp, Homo sapiens
  CDS (92)..(3163)
ctcctccctcctcccttgctcgctcgctggctccctccccccgggccggctcggcgttgactccgccgcacgctgcagccgcggctggaagatggcggggaacgactgcggcgcgctgctggacgaagagctctcctccttcttcctcaactatctcgctgacacgcagggtggagggtccggggaggagcaactctatgctgactttccagaacttgacctctcccagctggatgccagcgactttgactcggccacctgctttggggagctgcagtggtgcccagagaactcagagactgaacccaaccagtacagccccgatgactccgagctcttccagattgacagtgagaatgaggccctcctggcagagctcaccaagaccctggatgacatccctgaagatgacgtgggtctggctgccttcccagccctggatggtggagacgctctatcatgcacctcagcttcgcctgccccctcatctgcaccccccagccctgccccggagaagccctcggccccagcccctgaggtggacgagctctcactgctgcagaagctcctcctggccacatcctacccaacatcaagctctgacacccagaaggaagggaccgcctggcgccaggcaggcctcagatctaaaagtcaacggccttgtgttaaggcggacagcacccaagacaagaaggctcccatgatgcagtctcagagccgaagttgtacagaactacataagcacctcacctcggcacagtgctgcctgcaggatcggggtctgcagccaccatgcctccagagtccccggctccctgccaaggaggacaaggagccgggtgaggactgcccgagcccccagccagctccagcctctccccgggactccctagctctgggcagggcagaccccggtgccccggtttcccaggaagacatgcaggcgatggtgcaactcatacgctacatgcacacctactgcctcccccagaggaagctgcccccacagacccctgagccactccccaaggcctgcagcaacccctcccagcaggtcagatcccggccctggtcccggcaccactccaaagcctcctgggctgagttctccattctgagggaacttctggctcaagacgtgctctgtgatgtcagcaaaccctaccgtctggccacgcctgtttatgcctccctcacacctcggtcaaggcccaggccccccaaagacagtcaggcctcccctggtcgcccgtcctcggtggaggaggtaaggatcgcagcttcacccaagagcaccgggcccagaccaagcctgcgcccactgcggctggaggtgaaaagggaggtccgccggcctgccagactgcagcagcaggaggaggaagacgaggaagaagaggaggaggaagaggaagaagaaaaagaggaggaggaggagtggggcaggaaaaggccaggccgaggcctgccatggacgaagctggggaggaagctggagagctctgtgtgccccgtgcggcgttctcggagactgaaccctgagctgggcccctggctgacatttgcagatgagccgctggtcccctcggagccccaaggtgctctgccctcactgtgcctggctcccaaggcctacgacgtagagcgggagctgggcagccccacggacgaggacagtggccaagaccagcagctcctacggggaccccagatccctgccctggagagcccctgtgagagtgggtgtggggacatggatgaggaccccagctgcccgcagctccctcccagagactctcccaggtgcctcatgctggccttgtcacaaagcgacccaacttttggcaagaagagctttgagcagaccttgacagtggagctctgtggcacagcaggactcaccccacccaccacaccaccgtacaagcccacagaggaggatcccttcaaaccagacatcaagcatagtctaggcaaagaaatagctctcagcctcccctcccctgagggcctctcactcaaggccaccccaggggctgcccacaagctgccaaagaagcacccagagcgaagtgagctcctgtcccacctgcgacatgccacagcccagccagcctcccaggctggccagaagcgtcccttctcctgttcctttggagaccatgactactgccaggtgctccgaccagaaggcgtcctgcaaaggaaggtgctgaggtcctgggagccgtctggggttcaccttgaggactggccccagcagggtgccccttgggctgaggcacaggcccctggcagggaggaagacagaagctgtgatgctggcgccccacccaaggacagcacgctgctgagagaccatgagatccgtgccagcctcaccaaacactttgggctgctggagaccgccctggaggaggaagacctggcctcctgcaagagccctgagtatgacactgtctttgaagacagcagcagcagcagcggcgagagcagcttcctcccagaggaggaagaggaagaaggggaggaggaggaggaggacgatgaagaagaggactcaggggtcagccccacttgctctgaccactgcccctaccagagcccaccaagcaaggccaaccggcagctctgttcccgcagccgctcaagctctggctcttcaccctgccactcctggtcaccagccactcgaaggaacttcagatgtgagagcagagggccgtgttcagacagaacgccaagcatccggcacgccaggaagcggcgggaaaaggccattggggaaggccgcgtggtgtacattcaaaatctctccagcgacatgagctcccgagagctgaagaggcgctttgaagtgtttggtgagattgaggagtgcgaggtgctgacaagaaataggagaggcgagaagtacggcttcatcacctaccggtgttctgagcacgcggccctctctttgacaaagggcgctgccctgaggaagcgcaacgagccctccttccagctgagctacggagggctccggcacttctgctggcccagatacactgactacgattccaattcagaagaggcccttcctgcgtcagggaaaagcaagtatgaagccatggattttgacagcttactgaaagaggcccagcagagcctgcattgataacagccttaaccctcgaggaatacctcaatacctcagacaaggcccttccaatatgtttacgttttcaaagaaatcaagtatatgaggagagcgagcgagcgtgagagaacacccgtgagagagacttgaaactgctgtcctttaaaaaaaaaaaaaatcaatgtttacattgaacaaagctgcttctgtctgtgagtttccatggtgttgacgttccactgccacattagtgtcctcgcttccaacgggttgtcccgggtgcacctcgaagtgccgggtccgtcacccatcgccccttccttcccgactgacttcctctcgtagacttgcagctgtgttcaccataacatttcttgtctgtagtgtgtgatgatgaaattgttacttgtgaatagaatcaggactataaacttcatttttaattgaaaaaaaaagtatatccttaaaataatgtatttatggctcagatgtactgtgcctgggattattgtattgcttccttgattttttaactatgcactgtcatgaggtgtttgccactgagctgccctgctccccttgccagattgccctggaggtgctgggtggccgctaggctggtctgcaggaaagcgcggcctgccgtttccgggccgtatctgccaagccctgccttgtctcttactgagcaagtttggctcaaattataggagcccccatcttgtgcccagctcatgctccaagtgtgtgtctatccatttgtactcagactcttgagtaccttgtaaggaaggcggggcaagctgcatcattcctgttttccaggggaggctggcagctcctcaagaggcgaaatgactgtgggaggtccggttaccagtgaggaggcagagcggtgacccagaccaggccttctggttcttggtcccgtgcttccgtagtagctggggtaaagacaccgtttcagggactggtagaggtgagttcggctaaattgggcaccgggctagaagcctaagggctcattttaggggttacattaggtgttgattcaccagcatcaggtgaattcaagccctggcatgtgtcttggatgcaccatcagctttgatcctgagtggtcctgcggtttgtctgtgcctgtggacacactgtcagaacttcagtgacacccctggcagcggtacagacaggtggtctgggagcagtcatcttttttgggccagccaccagcccatcctactccctcaggtagtccttcgtctttaccttgtccttgtctgtaaagttgttttggtggctggggcaggggagccaggaggagggagtgaaggttgggaatagataggacaatctcctagctctcctccaattgagaaaacactccaattgggctttgctttaaactttgtgttcttaagtgatgtcaaagccatttccagcttaatgttctgtgggtaccttgggggccattcatgcagggagcatggccaggcagggtatgagtacattgtttctgatttctttcatacatcagggttcctcgggaaatttttgtattttttttttaagtcctgctgctttaaaaatttgaaagtggctcattaaactaaacaggctaatgtaatttgttgcttatgccaagcctagactgttgagaattgacgtttttaaagattatcaaatacctcagtaggtaaaatgagcccatgatcttccactgagtggtgagcatactcccagcccatggacaaggccggaagagacaggctttagtaggggtagggaatttgaactgttgtgtgtcacagcagttgacctctctggactccaatttcctttcctgtgaaatgaactgattagacatgtttcaacattgttagcttctgctgaggcagtgtctagcccaagatggcaaatacatagctcatgtgccactactcccacctccttgaccaatacagacataactaatcaatcacaccactcaggttccctgagcctggatgtgctataagaatcctgaaatcagtgctctggtaagtcattactaattgattagagttcaatctatttgacatcttgggctaatctttggaaggtttccaacaatcacacaaaaccatatgctggctgggtttcatgctggcctatccctgtctgtgatgttccgttccatgagagaaaactcccctaatgctattccatggcgtaacactcccaatactattttgacgcccacgtccccttgcagagggtgcagggggcggtagacgaatgacagacaggaacatatttggggaaggcagggcttaggaagatggaccaaaaagggacttcccacagcacagacctgatcattcggatttcctctttagctattcactgcctagcacatagtaggcacacaataaatgattatggaatgggataaaatttagatctttctgctgcctccactaagttaagtcctgatttacatcaaggagagaactgagataggaaagaacactagattccaagtctggagagttgggggagtccagattctaccaagaatttcctttgtaactttggtaagtcccttttactccctggcaccccggtgtgctgaaaggagttggtccatatatgatctcttagcccctcctatttgcttcttccttgattgctcttggtcaaagggtcagccttgggctggtgatactttagagtaaagaaatggagagttttagcaaaggaccagtctgtccctccctgctttggggtcagctaaagctgtcctttcatgtcagattaacctaggacacttgtagttagcttagacgttggcccttgagcagagacctgagcgtggcattgggacatgacatacctaaagtcagggctaggggacgctgcctgccaagggcatcgagtagtctctacttgctatcccgtacataaaatgctacaagttctaaaatttaccgaccctgcagacaacctctatcccgaaggactcattcggtgctgtgtattatttagggcaactccaaggtctattcagaaaaacgagtgaaccttggtctctttcccaccaaattgaggagtaacccagagggagcagctgccattggcaaccatctcgttgtagctctgtcctagtgtttgctcttgatgatgtttacatgtgatcgccataaagcttgctgtagactgtgtcgatagccgcccgcacagggcaggtcgtactgtccgtttctgtgccgtgctggtgttttccaaaaatgtctgatccaaccactaagtggaattcttccatctccttcctcagtctgtacaaggctgaatcagaatccccattctcgggggctctggttaccgaaggaaaatgcatcaaagagttaaagaatatgagtggatggagtgcagctaaggcccccaccccctgctccgtcacaacttgccccctcaaccaaaaagctgctttgagtcaaaaagcacccataagatacctgcatctgccttgaaatcttgcagcatggagtgtcatatgtactcaggagagaggcagggctttgcgggcaggagaaggaagggaggaatgctctgagctgcaaagacccagtactcaagttctgacgtgggaggagatgcagtgagacgtctcttgttgcctaaagcctgttcctgttggttttcttagagtgatttctcctagacatgtgcagtaggcccactggggctgctgtgcagtggtgagtaaaagggcagggaaggcatggacagcctggtccttctgcatggacagctcagtccatggcccatcccaggtatagagttcagttaatcccatttgagcctgcagcttaagagatggctcatcctaactgtgaagcaaaatcagccccagaggatgtattgatctgactcactgatgtcaaaattgcagtatttttttagcatttgagatttagcagctgccttcagtttggggttacccacatcccagcatcagatatgattaaggaaagaaattggatgtacaacagcaaagaaagtgaatgtcatggtttccctggccaaagaagagggaccctgtcatccttaccaatggggaagaagaaaactagtgcatgtgcaatatgtcaaagttagtcccctagtccctgaggggtttttacacacagatgggctccaggtctgctcgtcaagtttggaggtaccgggtaaatggaggggagctgcagagttggaaacccacatgcatggatgtgtccttggcccagaaccaccatgggatgggggaggccctgagccggctacaagacacccaggaagtaggcaaaggctgactttgcattaaacaataaaagcactttgagaaaaccccaacacttcagcctgggtccgtgtttctacactggaaaatacgagtctcctttggctgtgtgaagtgatcttctagagactgggacagggagtttgggaatggggctgctgtcaggtaggagagagcagagatgcctttggagatgtcagcagcaggagagccagtgctggggccaaccctttgctggccttttgttggaagcccttgaaacagggagccatgggtttagatcttggtacctacctttacagaaagatgaaaacagcccagctgagtgaaatgagtttgtagagtaagtcacttaactgtaagccatctcagaatcagaaaccctaatgtttcttacttgctatgtgaccttgggcccctgtttcctcatctaccaaatgagaatgttgaatatgagcattaaagtccctttcacctctgagaggctcagatccccaaccaggagcattgggaatccatcactcctccttgaaactgattccattctctgacttgacccagctcctgttcagggtgagggttctctgcaagaaccaaccagcagtaggttcaatcccactgtgtcctggctgagttgccttatccaagaagaccagctccccgggacagatctaagccatagtttctagtggggacagtaaggaattaaacccccaacttggctaggtaacgatgtcaaatctcacattaaccttgtctttgtccccactggatagctgttaatccgaatgttgtgaccatttggctgtttctctcttgttctcagacaatactagcaatacactttttttttttttttttaaagaaaaacagcttaggagcttttcacacatttctttcaaatgattgtaaaacatatggggcaacaggaggcattgatcgcgctgcatatgtttagggcagcttttgttttttgtttctttaatggtatagcagcagtgactgagccttcgtgattcctggggacagcttttcagatactctgtttcatcagtatgctttgcacatccggaaggagtacaaaaatccaactgcccaaatttggggcttggaaaataggttttataggtggtcggtccctgggctgtgcaacaactcctcaaagaggggtttatataactagaacccccctgggctgtatttttggtcaaaggagtctccaaggcggcttacaaaagcttcctttttcacttgaccacccttgctcattggttacttgtgaagggaattggtcagtttccacctcagcactttgccttatcaacatgcggtcgccatctagtggccaaaggttgtctccaccagctacccagatggaaggcaaataaatcctttcggccaccctgctgtccatcgtgaactttgggaatgaaatataatggcctgaacgaactgcctttgtgttcagagatcagtgcaacactagggtcagaagactccagaagcagccacttagtagactctcacgcagaactgagaaatgcactagctgtcctgtgggcagaagagacaggagtggaccaggagaggtccaggtgcccgggaagggtttactgtaactgcaatactggcagcccagctgctgaccttgttaagtaaacctttgctgggtggtccgaattctgccctcaaggcaagataagaagttgggtgtaaggattttgtggggggcctggccatgatctttgatatgatccccgaatagccaaatagttttttttgttcaattttttgtttctgtattttgtatttttaaaatcttgtcaaatgtttttgtgttaggaataaaaagtcataaactattcccaactttgtttcttgagggatgttctgattccaatggaaacaggtgggaaatctcaaggggagcgtggacaaggtggtatgtgcagcaggggaatagactgcttggatttccaaatggtttctggggaagatgaccatccagaagtccagcttagtgcagtctgctctggaattcacacccaccccctcgcctccttgtgccatgttgttagcattggcttggagcatctgcttcttccagaggcagctgctaatgttgaaaccaacacgagccctctccccaaccccaggtttctaaagaaggtgtctgtagccagccttaatcaactgggcaaggtggtccctatggtcctttccagcatttccaaatcttggactcaaattattttctcttggtgtgaccacacagcctagagaattctgagcaataggagccagggctttccctgactctgcgacagggtcaaaccaaggaatggctaaacctgtgaggttttgtcatccccgggggtactactgtagggggcattatttattaggaagcttaacaaggtaactacggcctgagtgcgtgagtgtaaggctgtgtttgtggtgggggtgtgtgtgtgtgtatctgtgcacacatacacacgtctgtgcctgtgtgtgtgtgtttgtgtgtgtgtgtgtgtgtggaattacattgatgcatttattgagaaaggtgcaagaatttcacctacacagagggacacatctgctttgttatttataatagaaagctaaattttaattttttaaaggacactgctaatgattgagaatcaagtttttagttttgctattttttttaattggtagaggatttttatatattttttccattttgttgggttgtgtccttatttatataaatactttatccgtaagaggcaaggaggaaaccttctttgcttttacatattgtggttgtcatcgtccctattttatttctggtgtgatttctctgtcttaccttctaaatgagaaaatgttttcttgtatttgtacattgtcagattctatagtttcctagataatttaaccaaattgctctatgtattattattctgtgagtataaagttctattttaatgtctgtaaatacttcagaactggcttcttttctcaaactcccactgtggggttattgtttacatcacagaaactgtagaatctctatgctcatgtactgtaaatagtgaagtgatctgcttataaataaacttaacaaatacactatggagattaaaaacaaaataccacccacaaaaaaaaaaaaaaaa

SEQ ID NO: 10 - protein, 1023 aa, Homo sapiens
MAGNDCGALLDEELSSFFLNYLADTQGGGSGEEQLYADFPELDLSQLDASDFDSATCFGELQWCPENSETEPNQYSPDDSELFQIDSENEALLAELTKTLDDIPEDDVGLAAFPALDGGDALSCTSASPAPSSAPPSPAPEKPSAPAPEVDELSLLQKLLLATSYPTSSSDTQKEGTAWRQAGLRSKSQRPCVKADSTQDKKAPMMQSQSRSCTELHKHLTSAQCCLQDRGLQPPCLQSPRLPAKEDKEPGEDCPSPQPAPASPRDSLALGRADPGAPVSQEDMQAMVQLIRYMHTYCLPQRKLPPQTPEPLPKACSNPSQQVRSRPWSRHHSKASWAEFSILRELLAQDVLCDVSKPYRLATPVYASLTPRSRPRPPKDSQASPGRPSSVEEVRIAASPKSTGPRPSLRPLRLEVKREVRRPARLQQQEEEDEEEEEEEEEEEKEEEEEWGRKRPGRGLPWTKLGRKLESSVCPVRRSRRLNPELGPWLTFADEPLVPSEPQGALPSLCLAPKAYDVERELGSPTDEDSGQDQQLLRGPQIPALESPCESGCGDMDEDPSCPQLPPRDSPRCLMLALSQSDPTFGKKSFEQTLTVELCGTAGLTPPTTPPYKPTEEDPFKPDIKHSLGKEIALSLPSPEGLSLKATPGAAHKLPKKHPERSELLSHLRHATAQPASQAGQKRPFSCSFGDHDYCQVLRPEGVLQRKVLRSWEPSGVHLEDWPQQGAPWAEAQAPGREEDRSCDAGAPPKDSTLLRDHEIRASLTKHFGLLETALEEEDLASCKSPEYDTVFEDSSSSSGESSFLPEEEEEEGEEEEEDDEEEDSGVSPTCSDHCPYQSPPSKANRQLCSRSRSSSGSSPCHSWSPATRRNFRCESRGPCSDRTPSIRHARKRREKAIGEGRVVYIQNLSSDMSSRELKRRFEVFGEIEECEVLTRNRRGEKYGFITYRCSEHAALSLTKGAALRKRNEPSFQLSYGGLRHFCWPRYTDYDSNSEEALPASGKSKYEAMDFDSLLKEAQQSLH

SEQ ID NO: 11 - DNA, 3656 bp, Mus musculus
  CDS (65)..(3109)
ctcgctccctcccccgggcgggctcggcgctgactccgccgcacgctgcagccgcggctggaagatggcggggaacgactgcggcgcgctgctggatgaagagctctcgtccttcttcctcaactatctctctgacacgcagggtggggactctggagaggaacagctgtgtgctgacttgccagagcttgacctctcccagctggacgccagtgactttgactcagccacgtgctttggggagctgcagtggtgcccggagacctcagagacagagcccagccagtacagccccgatgactccgagctcttccagattgacagtgagaatgaagctctcttggctgcgcttacgaagaccctggatgacatccccgaagacgatgtggggctggctgccttcccagaactggatgaaggcgacacaccatcctgcaccccagcctcacctgcccccttatctgcaccccccagccccaccctggagaggcttctgtccccagcgtctgacgtggacgagctttcactgctacagaagctcctcctggccacatcctccccaacagcaagctctgacgctctgaaggacggggccacctggtcccagaccagcctcagttccagaagtcagcggccttgtgtcaaggtggatggcacccaggataagaagacccccacactgcgggctcagagccggccttgtacggaactgcataagcacctcacttcggtgctgccctgtcccagagtgaaagcctgctccccaactccgcacccgagccctcggctcctctccaaagaggaggaggaggaggtgggggaggattgcccaagcccttggccgactccagcctcgccccaagactccctagcacaggacacggccagccccgacagtgcccagcctcccgaggaggatgtgagggccatggtacagctcattcgctacatgcatacctactgcctgcctcagaggaagctgccccaacgggccccagagccaatcccccaggcctgcagcagcctctccaggcaggttcaaccccgatcccggcatccccccaaagccttctggactgagttctctatcctaagggaacttctggcccaagatatcctctgtgatgttagcaagccctaccgcctggccatacctgtctatgcttccctcacacctcagtccaggcccaggccccccaaggacagtcaggcctcccctgcccactctgccatggcagaagaggtgagaatcactgcttcccccaagagcaccgggcctagacccagcctgcgtcctctgaggctggaggtgaaacgggatgttaacaagcctacaaggcaaaagcgggaggaagatgaggaggaggaggaggaagaagaagaagaggaagaagaaaaagaagaggaagaagaggagtggggcaggaagagaccaggtcgtggcctgccatggaccaaactagggaggaagatggacagctccgtgtgccccgtgcggcgctccaggagactgaatccagagctgggtccctggctgacattcactgatgagcccttaggtgctctgccctcgatgtgcctggatacagagacccacaacctggaggaagacctgggcagcctcacagacagtagtcaaggccggcagctcccccagggatcccagatccccgccctggaaagcccctgtgagagtgggtgcggagacacagatgaagatccaagctgcccacagcccacttccagagactcctccaggtgcctcatgctggccttgtcacaaagcgactctcttggcaagaagagctttgaggagtccctgacggtggagctttgcggcacggcaggactcacgccacccaccacacctccatacaagccaatggaggaggaccccttcaagccagacaccaagctcagcccaggccaagacacagctcccagccttccctcccccgaggctcttccgctcacagccaccccaggagcttcccacaagctgcccaagaggcacccagagcgaagcgagctcctgtcccatttgcagcatgccacaacccaaccagtctcacaggctggccagaagcgccccttctcctgctcctttggagaccacgactactgccaggtgctcaggccagaggctgccctgcagaggaaggtgctgcggtcctgggagccaatcggggtccaccttgaagacttggcccagcagggtgcccctctgccaacggaaacaaaggcccctaggagggaggcaaaccagaactgtgaccctacccacaaggacagcatgcagctaagagaccatgagatccgtgccagtctcacaaagcactttgggctgctggagactgctctggaaggtgaagacctggcgtcctgtaaaagcccggagtatgacaccgtatttgaggacagcagcagcagcagtggcgagagtagcttcctgcttgaggaggaggaggaagaggaggagggaggggaagaggacgatgaaggagaggactcaggggtcagccctccctgctctgatcactgcccctaccagagcccacccagtaaggccagtcggcagctctgctcccgaagccgctccagttccggctcctcgtcctgcagctcctggtcaccagccacccggaagaacttcagacgtgagagcagagggccctgttcagatggaaccccaagcgtccggcatgccaggaagcggcgggaaaaggccatcggtgaaggccgtgtggtatacattcgaaatctctccagtgacatgagctctcgggaactaaagaagcgctttgaggtgttcggtgagattgtagagtgccaggtgctgacgagaagtaaaagaggccagaagcacggttttatcaccttccggtgttcagagcacgctgccctgtccgtgaggaacggcgccaccctgagaaagcgcaatgagccctccttccacctgagctatggagggctccggcacttccgttggcccagatacactgactatgatcccacatctgaggagtcccttccctcatctgggaaaagcaagtacgaagccatggattttgacagcttactgaaagaggcccagcagagcctgcattgatatcagccttaaccttcgaggaatacctcaatacctcagacaaggcccttccaatatgtttacgttttcaaagaaaagagtatatgagaaggagagcgagcgagcgagcgagcgagcgagtgagcgtgagagatcacacaggagagagaaagacttgaatctgctgtcgtttcctttaaaaaaaaaaaaacgaaaaacaaaaacaaatcaatgtttacattgaacaaagctgcttccgtccgtctgtccgtccgtccgtccgtccgtgagtttccatgctgttgatgttccactgccacgttagcgtcgtcctcgcttccagcggatcgtcctgggtgcgcctccaagtgctgtcagtcgtcctctgcccctcccacccgactgacttccttctgttagacttgagctgtgttcacataacatcttctgtctgtagagtgtgatgatgacattgttacttgtgaatagaatcaggagttagaaactcatttttaattgaagaaaaaaaaagtatatccttaaaaagaaaaaaaaaaaaacaaatgta

SEQ ID NO: 12 - protein, 1014 aa, Mus musculus
MAGNDCGALLDEELSSFFLNYLSDTQGGDSGEEQLCADLPELDLSQLDASDFDSATCFGELQWCPETSETEPSQYSPDDSELFQIDSENEALLAALTKTLDDIPEDDVGLAAFPELDEGDTPSCTPASPAPLSAPPSPTLERLLSPASDVDELSLLQKLLLATSSPTASSDALKDGATWSQTSLSSRSQRPCVKVDGTQDKKTPTLRAQSRPCTELHKHLTSVLPCPRVKACSPTPHPSPRLLSKEEEEEVGEDCPSPWPTPASPQDSLAQDTASPDSAQPPEEDVRAMVQLIRYMHTYCLPQRKLPQRAPEPIPQACSSLSRQVQPRSRHPPKAFWTEFSILRELLAQDILCDVSKPYRLAIPVYASLTPQSRPRPPKDSQASPAHSAMAEEVRITASPKSTGPRPSLRPLRLEVKRDVNKPTRQKREEDEEEEEEEEEEEEEKEEEEEEWGRKRPGRGLPWTKLGRKMDSSVCPVRRSRRLNPELGPWLTFTDEPLGALPSMCLDTETHNLEEDLGSLTDSSQGRQLPQGSQIPALESPCESGCGDTDEDPSCPQPTSRDSSRCLMLALSQSDSLGKKSFEESLTVELCGTAGLTPPTTPPYKPMEEDPFKPDTKLSPGQDTAPSLPSPEALPLTATPGASHKLPKRHPERSELLSHLQHATTQPVSQAGQKRPFSCSFGDHDYCQVLRPEAALQRKVLRSWEPIGVHLEDLAQQGAPLPTETKAPRREANQNCDPTHKDSMQLRDHEIRASLTKHFGLLETALEGEDLASCKSPEYDTVFEDSSSSSGESSFLLEEEEEEEEGGEEDDEGEDSGVSPPCSDHCPYQSPPSKASRQLCSRSRSSSGSSSCSSWSPATRKNFRRESRGPCSDGTPSVRHARKRREKAIGEGRVVYIRNLSSDMSSRELKKRFEVFGEIVECQVLTRSKRGQKHGFITFRCSEHAALSVRNGATLRKRNEPSFHLSYGGLRHFRWPRYTDYDPTSEESLPSSGKSKYEAMDFDSLLKEAQQSLH

SEQ ID NO: 13 - DNA, 19 bp, Mus musculus
cttgctgagtcagcacccc

SEQ ID NO: 14 - DNA, 19 bp, Mus musculus
catgctgactcagcagctc

SEQ ID NO: 15 - DNA, 41 bp, Mus musculus
acgctcctgccgctgagctgtgactcagccagagaactgag

SEQ ID NO: 16 - DNA, 42 bp, Mus musculus
cacaccctgcccagaggcacacttgctgagtcagcaccccgg

SEQ ID NO: 17 - DNA, 42 bp, Mus musculus
ttgatagtgaggggaacatgctgactcagcagctccgaataa

SEQ ID NO: 18 - DNA, 19 bp, Homo sapiens
gggattcggtgaagatttg

SEQ ID NO: 19 - DNA, 30 bp, Artificial sequence
  synthetic DNA
agccatatgggctgcagtcaggacttctcc

SEQ ID NO: 20 - DNA, 30 bp, Artificial sequence
  synthetic DNA
atcctcgagtcacttcctccggtcctttgg

SEQ ID NO: 21 - DNA, 20 bp, Artificial sequence
  synthetic DNA
taatacgactcactataggg

SEQ ID NO: 22 - DNA, 38 bp, Artificial sequence
  synthetic DNA
ctttatgtttttggcgtcttccttcctccggtcctttg

SEQ ID NO: 23 - DNA, 38 bp, Artificial sequence
  synthetic DNA
caaaggaccggaggaaggaagacgccaaaaacataaag

SEQ ID NO: 24 - DNA, 20 bp, Artificial sequence
  synthetic DNA
gactctagaattacacggcg

SEQ ID NO: 25 - DNA, 20 bp, Artificial sequence
  synthetic DNA
taatacgactcactataggg

SEQ ID NO: 26 - DNA, 39 bp, Artificial sequence
  synthetic DNA
ctttatgtttttggcgtcttccatctgcttgtccaggaa

SEQ ID NO: 27 - DNA, 39 bp, Artificial sequence
  synthetic DNA
ttcctggacaagcagatggaagacgccaaaaacataaag

SEQ ID NO: 28 - DNA, 20 bp, Artificial sequence
  synthetic DNA
gactctagaattacacggcg